(19) United States
(12) Patent Application Publication
(10) Pub. No.: US 2008/0044437 A1
(43) Pub. Date: Feb. 21, 2008

(54) Encapsidation System for Production of Recombinant Virus-Like Particles

(76) Inventor: Qun Chen, San Francisco, CA (US)

Correspondence Address: Medlen&Carroll, 101 Howard Street Suite 350, SanFrancisco, CA, 94105, US

(21) Appl. No.: 11/661,912

(22) PCT Filed: Sep. 1, 2005
(86) PCT No.: PCT/US05/31520

§ 371 Date: Aug. 17, 2007

Related U.S. Application Data
(60) Provisional application No. 60/607,240, filed on Sep. 2, 2004.

(51) Int. Cl. A61K 39/12; A61P 37/00; C12N 15/86

(52) U.S. Cl. 424/204.1; 435/69.1

(57) ABSTRACT

The present invention relates to methods and compositions for manufacturing virus-like particles (VLPs), suitable for use as immunogens and as research tools. The VLPs of the present invention provide a sale alternative to the use of pathogenic viruses for clinical and laboratory applications.

### FIELD OF THE INVENTION

The present invention relates to methods and compositions for manufacturing virus-like particles (VLPs), suitable for use as immunogens and as research tools. The VLPs of the present invention provide a safe alternative to the use of pathogenic viruses for clinical and laboratory applications.

### BACKGROUND OF THE INVENTION

Developing more effective prophylactic and therapeutic strategies against highly mutable viruses, which infect crucially important populations of cells poses one of the major medical challenges of this century (See, e.g., Neumann et al, J Gen Virol. 83:2635-62, 2002; Wilhelmi et al., Clin Microbiol Infect, 9:247-62, 2003; Piedra, Pediatr Infect Dis J, 22(2 Suppl):S94-9, 2003; Gallo, Immunol Rev, 185:236-65, 2002; and Bangham, J Clin Pathol, 53:581-6, 2000). This is especially true for the immunodeficiency viruses that integrate into the host genome (See, e.g., Walker and Korber, Nat Immunol, 2:473-475, 2001), the coronaviruses that incorporate genes from other viruses (Farsang et al, Avian Pathol, 31:229-36, 2002; and Herrewegh et al, J Virol, 72:4508-14, 1998), and the influenza viruses that recombine genetic segments between different viral strains (Reid and Taubenberger, J Gen Virol. 84:2285-92, 2003). The genetic variation seen in the genomes of these viruses is the result of mutation, recombination, insertion, deletion, and/or reassortment (See, e.g., Menendez-Arias et al, Curr Drug Targets Infect Disord. 3:355-71, 2003). These processes in turn result in an extraordinary degree of antigenic variability, permitting immune evasion and continued infection. Numerous preventative and therapeutic vaccine strategies and modalities have been explored including: 1) inactivated or whole killed viruses; 2) live attenuated viruses; 3) subunit vaccines; 4) live vectors (e.g., BCG, yellow fever virus, poxvirus, polio virus, adenovirus, Salmonella, etc.); 5) peptide epitope vaccines; 6) DNA vaccines; 7) liposomes and 8) virus-like particles and pseudovirions (Kaur and Johnson, 2003, HIV Med, 11:76-85, 2003; Glansbeek et al., J Gen Virol, 2002 83:1-10, 2002; Cavanagh, Avian Pathol, 32:567-82, 2003; Monath, Ann NY Acad Sci, 951:1-12, 2001; and Koff, Int J Parasitol, 33:517-23, 2003).

To date, many viruses remain a threat to human populations due to the lack of efficacious vaccines, for instance, the human immunodeficiency virus (HIV, McMichael and Hanke, Nat Med, 9: 874-880, 2003; and Klein, Vaccine 21: 616-619, 2003), severe acute respiratory syndrome coronavirus (SCV, Oxford et al, Immunology, 109:326-8, 2003), hepatitis C virus (HCV, Koff, Int J Parasitol, 33:517-23, 2003), some influenza viruses (Webby and Webster, Science, 302:1519-22, 2003), and Ebola virus (Watanabe et al, J Virol. 8:999-1005, 2004). Decades after their introduction, complications continue to arise from the use of attenuated viruses or “vaccine” strains of polio and influenza, leading to increased safety concerns (Kemble and Greenberg, Vaccine, 21:1789-95, 2003; and Dowdle et al, Rev Med Virol, 13:277-91, 2003). Examples of viral infections by “vaccine” strains include avian coronavirus vaccine “Massachusetts strain” (Farsang et al., Avian Pathol, 31:229-36, 2002), Polio vaccine “Sabin strain” (Kew et al., Science, 296:356-359, 2002; and Nathanson and Fine, Science, 296:269-270, 2002), measles-mumps-rubella vaccine (Uhlmann al., Mol Pathol, 55:84-90, 2002), Vaccinia (Griffiths, Rev Med Virol, 13:69-70, 2003), Influenza vaccine “FluMist” (Harper et al., MMWR Recomm Rep, 52(RR-13):1-8, 2003), and bovine viral diarrhea virus vaccine 1741 (Becher et al., J Virol, 75:6256-64, 2001). Thus, there remains an urgent need for the development of effective methods and compositions for combating viral infections.

### SUMMARY OF THE INVENTION

The present invention relates to methods and compositions for manufacturing virus-like particles (VLPs), suitable for use as immunogens and as research tools. The VLPs of the present invention provide a safe alternative to the use of pathogenic viruses for clinical and laboratory applications.

In particular, the present invention provides methods for producing a virus-like particle, comprising the steps of: providing: i) an encapsidation system comprising at least one packaging vector and two or more helper vectors, wherein the packaging vector comprises a nucleic acid having a packaging signal(s) but lacking an integration signal(s), and wherein the helper vectors each comprise one or more viral genes encoding one or more viral proteins, and ii) a cell line; introducing the encapsidation system into the cell line under conditions suitable for causing expression of the nucleic acid as a packaging signal and the one or more viral genes as one or more viral proteins, to produce a virus-like particle comprising one or more viral proteins assembled around the packaging signal; and purifying the virus-like particle. In some embodiments, the packaging vector further comprises a promoter/enhancer, and a terminator or polyadenylation site. In additional embodiments, the encapsidation vector further comprises a viral capsid gene in operable combination with the promoter/enhancer and the terminator or polyadenylation site. In some preferred embodiments, the at least two helper vectors comprises a first helper vector comprising a viral envelope or other structural protein gene, and second helper vector comprising a viral polymerase or other nonstructural gene. In other preferred embodiments, the at least two helper vectors further comprise one or more of a viral capsid gene, a viral regulatory gene, and a viral accessory gene. In particularly preferred embodiments, the virus-like particle is replication-deficient. In other particularly preferred embodiments, the virus-like particle is attachment and penetration competent. In some embodiments, penetration comprises fusion or endocytosis. In some preferred embodiments the virus-like particle is an immunodeficiency type virus-like particle. Ill preferred embodiments, the immunodeficiency type virus-like particle is an HIV-1 or HV-2 virus-like particle. The present invention also provides methods in which the encapsidation system comprises at least three helper vectors comprising a first helper vector comprising a viral spike gene, a second helper vector comprising a viral replicase gene, and a third helper vector comprising one or more viral subgenomic genes. In some preferred embodiments, the virus-like particle is a coronavirus type virus-like particle. In preferred embodiments, the coronavirus type virus-like particle is an SCV virus-like particle. In other preferred embodiments, the virus-like particle is a hepatitis type virus like particle. The present invention also provides methods in which the encapsidation system comprises at least eight helper vectors comprising a first helper vector having a viral polymerase A gene, a second helper vector having a viral polymerase B1 gene, a third helper vector comprising a viral polymerase B2 gene, a fourth helper vector comprising a viral nucleoprotein gene, a fifth helper vector comprising a viral membrane protein gene, a sixth helper vector comprising one or more viral nonstructural protein genes, a seventh helper vector comprising a viral hemagglutinin gene lacking a packaging signal and an eighth helper vector comprising a viral neuraminadase gene lacking a packaging signal. In some embodiments, the encapsidation system comprises a first packaging vector comprising a nucleic acid encoding a hemagglutinin packaging signal and a second packaging vector comprising a nucleic acid encoding a neuraminadase packaging signal. In some preferred embodiments, the virus-like particle is an influenza type virus-like particle.

Moreover, the present invention provides kits for producing of a virus-like particle, comprising: an encapsidation system comprising at least one packaging vector and two or more helper vectors, wherein the packaging vector comprises a nucleic acid having a packaging signal(s) but lacking an integration signal(s), and the at helper vectors each comprise one or more viral genes encoding one or more viral proteins, and instructions for cloning one or more viral genes lacking packaging signals into the helper vectors, and for contacting a suitable cell line with the encapsidation system under conditions suitable for causing expression of the nucleic acid as a packaging signal and the one or more viral genes as one or more viral proteins, to produce a virus-like particle comprising one or more viral proteins assembled around the packaging signal. In some embodiments, the kits farther comprise the suitable cell line. In particularly preferred embodiments, the encapsidation vector fiuther comprises a promoter/enhancer, and a terminator or polyadenylation site.

Additionally the present invention provides methods, comprising: providing: i) a subject; and ii) a composition comprising a virus-like particle; and administering the composition to the subject under conditions such that an immune response reactive with the virus-like particle is generated. In some embodiments, the immune response comprises one or more of a lymphocyte proliferative response, cytokine response, cytotoxic T lymphocyte response and antibody response. In some preferred embodiments, the cytokine response comprises secretion of one or more of an interleukin, interferon, tumor necrosis factor, chemokine, and growth factor. In particularly preferred embodiments, the antibody response comprises production of IgG antibodies and/or IgA antibodies. In some embodiments, the subject is a mammal selected from the group consisting of a human, a nonhuman primate, a horse, a cow, a sheep, a rodent, a goat and a cat. In other embodiments, the subject is a vertebrate animal selected from the group consisting of a fish, and a bird. In further embodiments, the subject is selected from the group consisting of an infected subject and an uninfected subject. In preferred embodiments, the composition is administered in a physiologically acceptable gas, solution or adjuvant, through at least one route selected from the group consisting of oronasal, intramuscular, intravenous, intraperitoneal, subcutaneous, oral, intranasal, intravaginal, intrarectal, and stomacheal.

The present invention also provides an encapsidation system suitable for producing a virus-like particle, the encapsidation system comprising: at least one packaging vector comprising a nucleic acid comprising less than 30% of a viral genome, the packaging vector having a packaging signal(s) but lacking an integration signal(s), and two or more helper vectors each comprising one or more viral genes encoding one or more viral proteins, wherein the one or more viral proteins are assembled around the packaging signal when the encapsidation system is introduced into a suitable cell line. Furthermore, the present invention provides a virus-like particle comprising one or more viral proteins assembled around a nucleic acid having a packaging signal(s) but lacking an integration signal(s), wherein the nucleic acid comprises less than 30% of a viral genome.

### DESCRIPTION OF THE FIGURES

FIG. 1 provides a schematic of the human immunodeficiency virus (HIV) type 1 proviral genome of the 89.6 isolate, with coding sequences of the HIV-1 genes depicted as open rectangles. This figure is adapted from U.S. patent application Ser. No. 10/207,346, herein incorporated by reference.

FIG. 2 provides a schematic comparison of HIV-1, HIV-2, simian immunodeficiency virus (SIV), and feline immunodeficiency virus (FIV) gene fragments, each containing leader sequences and a major packaging signal (ψ). This figure is adapted from Strappe et al., J Gen Virol, 84:2423-2430, 2003.

FIG. 3 depicts three recombinant packaging vector inserts. A first exemplary packaging vector comprises an HIV-1 nucleic acid sequence, a second exemplary packaging vector comprises an HIV-2 nucleic acid sequence, and a third exemplary packaging vector comprises a foreign (non-HIV) packaging sequence.Figure 4 depicts inserts of an exemplary HIV-1 encapsidation system comprising one packaging vector having an HIV-1 nucleic acid sequence, and three helper constructs, suitable for use in producing an exemplary HIV-1 virus-like particle (VLP). The packaging vector nucleic acid sequence, but not the nucleic acid sequences of the helper constructs, is packaged into the VLPs. The packaging signal of the packaging vector genome is denoted by A.

FIG. 5 depicts inserts of a second exemplary HIV-1 encapsidation system comprising one packaging vector having a foreign (non HIV) nucleic acid sequence, and three helper constructs, suitable for use in producing a second exemplary HIV-1 virus-like particle.

FIG. 6 depicts inserts of a third exemplary HIV-1 encapsidation system comprising one packaging vector having a foreign (non HIV) nucleic acid sequence, and three helper constructs, suitable for use in producing a third exemplary HIV-1 virus-like particle.

FIG. 7 provides two representative combinations of recombinant viral gene constructs suitable for use in producing two types of HIV-1 virus-like particles. The top particles comprise an HIV-1 nucleic acid sequence, and the bottom particles comprise a foreign (non HIV) nucleic acid sequence.

FIG. 8 provides a schematic of the life cycle of the two exemplary HIV-1 virus-like particles of FIG. 7. The VLPs bind to and gain entrance into the target cell, but do not produce HIV-1 virions.

FIG. 9 provides a schematic of the severe acute respiratory syndrome (SARS) coronavirus (SCV) genome of the BVP isolate, with coding sequences of the SCV genes depicted as open rectangles. FIG. 10 depicts inserts of an exemplary SCV encapsidation system comprising one packaging vector, and three helper constructs, suitable for use in producing a SCV virus-like particle of the present invention. The packaging vector insert comprises two packaging signals, one at the 5′end and the other in the SCV Rep 1b sequence.

FIG. 11 depicts three recombinant packaging vector inserts. A first exemplary packaging vector comprises both a first and a second packaging signal, a second exemplary packaging vector comprises only the first packaging signal, and a third exemplary packaging vector comprises only the second packaging signal.

FIG. 12 provides a schematic of multiple SCV replicase helper construct inserts on the left (1-3), and multiple SCV subgenomic helper construct inserts on the right (A-F).

FIG. 13 provides two representative combinations of recombinant viral gene constructs suitable for producing two types of SCV virus-like particles. The top encapsidation system comprises a packaging vector having two packaging signals, and five helper constructs, and the bottom encapsidation system comprises a packaging vector have a single packaging signal, and four helper constructs.

FIG. 14 provides a schematic of the hepatitis C virus (HCV) genome, and a summary of HCV polyprotein processing, with coding sequences of the HCV genes depicted as open rectangles. FIG. 15 depicts inserts of an exemplary HCV encapsidation system comprising one packaging vector, and two helper constructs, suitable for use in producing an exemplary HCV virus-like particle.

FIG. 16 depicts inserts of a second exemplary HCV encapsidation system comprising one packaging vector, and two helper constructs, suitable for use in producing a second exemplary HCV virus-like particle.

FIG. 17 depicts inserts of a third exemplary HCV encapsidation system comprising one packaging vector, and two helper constructs, suitable for use in producing a third exemplary HCV virus-like particle. In this embodiment, a foreign (non HCV) reverse transcriptase (RT) nucleic acid sequence is included in the two helper vector constructs, while the HCV 3′UTR has been omitted.

FIG. 18 provides a schematic of the eight gene segments of the influenza A virus (H1N1) genome of the A/WSN/33 isolate, with coding sequences depicted as open rectangles.

FIG. 19 depicts inserts of an exemplary influenza A virus (H1N1) encapsidation system comprising partial HA and NA packaging vectors, and HA and NA helper constructs (having mutated packaging signals), suitable for producing an exemplary influenza A virus (H1N1) virus-like particle.

FIG. 20 depicts inserts of an exemplary avian influenza virus (H5N1) encapsidation system comprising partial HA and NA packaging vectors, and HA and NA helper constructs (having mutated packaging signals), suitable for producing an exemplary avian influenza virus (H5N1) virus-like particle.

FIG. 21 depicts inserts of an exemplary influenza virus (H7N3) encapsidation system comprising partial HA and NA packaging vectors, and HA and NA helper constructs (having mutated packaging signals), suitable for producing an exemplary influenza virus (H7N3) virus-like particle.

### DEFINITIONS

To facilitate understanding of the invention, a number of terms are defined below.

The term “vaccine” as used herein, refers to a composition that is administered to produce or artificially increase immunity to a particular disease. For example, “vaccine compositions” frequently comprise a preparation of killed or live attenuated microorganisms. Alternatively, subunit vaccines frequently comprise a preparation of isolated nucleic acids or proteins corresponding to the genes or gene products of a microorganism of interest.

The terms “virus-like-particle,” “VLP,” “pseudovirus,” “pseudovirion,” and “letumsome” as used herein refer to a recombinant or synthetic particle comprising one or more viral proteins and a nucleic acid comprising a packaging signal, which facilitates the packaging (e.g, self assembly) of one or more viral proteins into a form resembling an intact virion. Specifically, the term “letumsome” refers to a synthetic VLP suitable for use as an immunogen. The term “letumsome” is derived from the Latin words: “letum” meaning ruin, annihilation, and death; and “some” meaning body, remains, and corpse. In particular the present invention provides virus-like particles comprising one or more viral proteins assembled around a nucleic acid having a packaging signal(s) but lacking an integration signal(s), wherein the nucleic acid comprises 90% to 0% of a viral genome. In preferred embodiments the nucleic acid comprises less than 60%, preferably less than 30%, more preferably less than 15%, and most preferably less than 7.5% of a viral genome (e.g., for an HIV VLP this figure is calculated as the number of HIV nucleotides packaged in the VLP divided by the number of HIV nucleotides packaged in an HIV virion of the same strain). In other preferred embodiments, the one or more viral proteins comprise 10% to 100% of the viral proteins. In particularly preferred embodiments, the one or more viral proteins comprise greater than 30%, preferably greater than 60%, more preferably greater than 90%, and most preferably greater than 99% of the viral proteins (e.g., for an HIV VLP this figure is calculated as the number of HIV amino acids assembled around the packaging signal(s) of the VLP divided by the number of HIV amino acids making up the viral proteins of an HIV virion of the same strain)

As used herein, the term “preventive vaccine” refers to a vaccine composition suitable for administration to an uninfected subject and which provides protection from microbial infection (e.g., sterilizing immunity) or reduces the severity of the microbial infection (e.g., reduced viral load). The term “therapeutic vaccine” refers to a vaccine composition suitable for administration to an infected subject and which prevents or delays microbial disease.

The term “subject” as used herein, refers to any animal (e.g., a mammal), including, but not limited to, humans, non-human primates, rodents, ovines, bovines, ruminants, lagomorphs, porcines, caprines, equines, canines, felines, aves, etc. Typically, the terms “subject” and “patient” are used interchangeably in reference to a human subject.

As used herein the term “mammal” refers to animals of the class Mammalia, including warm-blooded higher vertebrates such as placentals, marsupials, or monotremes, that nourish their young with milk secreted by mammary glands.

The term “at least” as used herein, refers to the minimum number or quantity suitable for various embodiments of the present invention. For instance, “at least four subunits ” refers to four or more subunits.

The term “immunodeficiency virus” refers to any lentivirus or to any member of the Lentivirus family that is capable of causing immune suppression in an infected animal. In some embodiments, the term “immunodeficiency virus” refers to the retrovirus known as the human immunodeficiency virus (HIV), which is responsible for the fatal illness termed the acquired immunodeficiency syndrome (AIDS). Two kinds of HI have been identified: HIV-1 is the more virulent, pandemic virus, and HIV-2 is a closely related virus, largely confmed to West Africa. The term “immunodeficiency virus” also refers to viruses causing immune suppression in nonhuman animals, including but not limited to SIV (primates), BIV and JDV (cattle), FIV (cats), MVV (sheep), OLV (sheep and goats), CAEV (goats), and EIAV (horses).

The terms “human immunodeficiency virus type-1” and “HIV-1” refer to the lentivirus that is widely recognized as the etiologic agent of the acquired immunodeficiency syndrome (AIDS). HIV-1 is characterized by its cytopathic effect and affinity for CD4+-lymphocytes and macrophages. The terms “human immunodeficiency virus type-2” and “HIV-2” refer to a lentivirus related to HIV-1 but carrying different antigenic components and with differing nucleic acid composition. The term “recombinant HIV strain” refers to an HIV virus produced from an immunodeficiency virus genome that has been assembled through the use of molecular biology techniques that are well known in the art. The terms “simian immunodeficiency virus” and “SIV” refer to lentiviruses related to HIV that cause acquired immunodeficiency syndrome in nonhuman primates (e.g., monkeys and apes). The terms “simian human immunodeficiency virus” and “SHIV” refer to various man-made chimeric retroviruses having both human and monkey immunodeficiency virus genes. The terms “feline immunodeficiency virus” and “FIV” refer to lentiviruses that cause acquired immunodeficiency syndrome in cats (e.g., cats and lions). The terms “bovine immunodeficiency virus” and “BIV” refer to lentiviruses that cause acquired immunodeficiency syndrome in cattle. Similarly, the terms “Jembrana disease virus” and “JDV” refer to lentiviruses distinct from BIV, but which also cause acquired immunodeficiency syndrome in cattle. The terms “equine infectious anemia virus” and “EIAV” refer to lentiviruses that cause acquired immunodeficiency syndrome in horses. The terms “caprine arthritis-encephalitis virus” and “CAEV” refer to lentiviruses that cause acquired immunodeficiency syndrome in goats. The terms “maedi-visna virus,” “visna virus” and “MVV” refer to lentiviruses that cause acquired immunodeficiency syndrome in sheep. The terms “ovine lentivirus” and “OLV” refer to lentiviruses that cause acquired immunodeficiency syndrome in goats and sheep.

The term “coronavirus” and “CoV” refer to any coronavirus or to any member of the Coronaviridae family that is capable of causing hepatitis, gastroenteritis, encephalitis, hypoxia, and/or pneumonia in an infected animal. In some embodiments, the term “SARS coronavirus” refers to the coronavirus known as the severe acute respiratory syndrome coronavirus (SCV), which is responsible for the fatal illness termed the severe acute respiratory syndrome (SARS). Three kinds of human coronaviruses have been identified: SCV is the more virulent, pandemic virus, human coronavirus 229E is causative agent of hepatitis, and human coronavirus OC43 is a closely related virus that causes encephalitis and gastroenteritis. The term “coronavirus” also refers to viruses causing pneumonia, hepatitis, gastroenteritis, and/or peritonitis in human and nonhuman animals, including but not limited to HCV 229E and HCV OC43 (human), BCV (cattle), FIPV (cats), PEDV, PTEV, PRV, PHEV (swine), MHV (mice), RSV (rats), and AIBV and TCV (birds).

The terms “severe acute respiratory syndrome coronavirus,” “SARS-CoV,” “SARSV,” “SCoV,” and “SCV” refer to the coronavirus that is widely recognized as the etiologic agent of the severe acute respiratory syndrome (SARS). SCV is characterized by its cytopathic effect and affinity for a metallopeptidase termed the angiotensin-converting enzyme 2 (ACE2) expressed in virtually all organs (Li et al., Nature, 426:450-4, 2003, and Hamming et al., J Pathol, 203:631-7, 2004). The terms “human coronavirus 229E” and “HCV-229E,” and “human coronavirus OC43” and “HCV-OC43,” refer to two coronaviruses related to SCV. The term “recombinant SCV strain” refers to a SARS virus produced from a severe acute respiratory syndrome coronavirus genome that has been assembled through the use of molecular biology techniques that are well known in the art. The terms “human coronavirus” and “HCoV” also refer to human coronaviruses related to SCV, but carrying different antigenic components and with differing nucleic acid composition. The terms “SCV-like coronavirus” and “SCV” refer to coronaviruses related to SCV that cause infection in wild mammals such as palm civets and nonhuman primates (Guan et al., Science, 302:276-8, 2003). The terms “bovine coronavirus” and “BCV” refer to coronaviruses that cause the bovine enteric and respiratory diseases in cattle. The terms “feline infectious peritonitis virus” and “FIPV” refer to coronaviruses that cause feline infectious peritonitis in cats (e.g., cats and lions). The terms “porcine epidemic diarrhea virus” and “PEDV” refer to coronaviruses that cause diarrhea in swine. Similarly, the terms “porcine transmissible gastroenteritis virus” and “TGEV,” “porcine hemagglutinating encephalomyelitis virus” and “PHEV,” “porcine respiratory coronavirus” and “PRV” refer to coronaviruses distinct from PDEV, which cause gastroenteritis, encephalomyelitis, and respiratory diseases respectively, in swine. The terms “mouse hepatitis virus,” “mouse coronavirus” and “MHV” refer to coronaviruses that cause hepatitis in mice. The terms “rat sialodacryoadenitis virus” and “RSV” refer to coronaviruses that cause sialodacryoadenitis in rats. The terms “avian infectious bronchitis virus” “avian coronavirus” and “IBV” refer to coronaviruses that cause bronchitis in wild birds and domestic poultry. Similarly, the terms “turkey coronavirus” and “TCV” refer to coronaviruses that cause bronchitis in birds and poultry. The terms “Bovine torovirus” and “BTV” refer to toroviruses that cause gastroenteritis in cattle. The terms “porcine torovirus” and “PTV” refer to toroviruses that cause gastroenteritis in swine. The terms “human torovirus” and “HTV” refer to toroviruses that cause gastroenteritis in human. The terms “equine torovirus” and “ETV” refer to toroviruses that are a causative agent of diarrhea in horses.

The terms “influenza virus” and “flu virus” refers to any influenza virus or to any member of the Orthomyxoviridae family, which is capable of causing coryza, cough, and myalgia in an infected animal. Three kinds of influenza viruses have been identified: influenza A virus is the annual outbreak pandemic virus; influenza B virus is more stable, causing outbreaks every 2-4 years; and influenza C virus is usually related to sporadic and subclinical infection. In some embodiments, the term “influenza A virus” refers to the influenza A virus known as the flu virus, which is responsible for the acute febrile and generally debilitating illness known as influenza disease. Influenza A virus is characterized by its cytopathic effect and affinity for cells with a sialic acid receptor. Influenza A viruses are divided into subtypes based on two proteins on the surface of the virus: hemagglutinin (H) and neuraminidase (N). There are 15 different hemagglutinin subtypes (H1-H15) and 9 different neuraminidase subtypes (N1-N9). Influenza A virus infects many different animals, including ducks, chickens, pigs, whales, horses, and seals. More than six HAs (H1, H2, H3, H5, H7, and H9) have been identified on viruses that infect humans. The terms “human influenza B virus” and “human influenza C virus” refer to two influenza viruses related to influenza A virus. The term “recombinant influenza A strain” refers to an influenza A virus produced from an influenza A genome that has been assembled through the use of molecular biology techniques that are well known in the art. The term “influenza virus” also refers to viruses causing respiratory diseases and conjunctivitis in human and nonhuman animals, and including but not limited to AH1N1 (birds, swine, and humans), AH3N2 (birds, marine mammals, and humans), AH2N2, AH5N1, and AH7N7 (birds and human), AH9N2 (birds and human), AH3N8 (birds and horses). The terms “avian influenza A virus” refer to influenza virus related to human influenza A virus that cause infection in wild birds (and sometimes humans) such as H1N1, H3N2, H5N1, and H7N7. The terms “swine influenza virus” refer to influenza virus related to influenza A virus that cause influenza in pigs. The terms “hemagglutinin,” “HA” and “H” refer to influenza A virus protein with subtypes from 1 to 15. The terms “neuraminidase,” “NA” and “N” refer to a second influenza A virus protein with subtypes from 1 to 9.

The terms “hepatitis C virus” and “HCV” refer to the hepatitis virus that is distantly related to flaviviruses and pestiviruses, which is widely recognized as the etiologic agent of hepatic fibrosis, cirrhosis, and hepatocellular carcinoma. Hepatitis C virus is characterized by its cytopathic effect and affinity for liver cells expressing DC-SIGN (dendritic cell-specific ICAM-3 grabbing nonintegrin; CD209) and L-SIGN (DC-SIGNR, liver and lymph node specific; CD209L) that function as HCV capture receptors, but do not mediate viral entry into target cells. Candidate HCV entry receptors include the: the scavenger receptor class B type1, the low-density lipoprotein receptor, and various glycosaminoglycans. The term “HCV subtypes” refers to viral isolates from different geographical regions that display significant genetic diversity. The term “HCV quasispecies” refers to different hepatitis C virus genotypes that coexist in infected individuals. The term “recombinant hepatitis C strain” refers to a hepatitis C virus produced from a hepatitis C viral genome that has been assembled through the use of molecular biology techniques that are well known in the art.

As used herein, the term “genome” refers to the total set of genes carried by an organism. In preferred embodiments, the term “genome” refers to the complete set of genes from a virus of interest. The term “gene” refers to a specific sequence of nucleotides (e.g., DNA or RNA) that is the functional unit of inheritance controlling the transmission and expression of one or more traits.

The terms “Gag,” “capsid,” “nucleocapsid,” “core,” “hemagglutinin,” “nucleoprotein,” “neuraminidase,” and “group specific antigen” refer various viral structural proteins (e.g., immunodeficiency virus polyprotein composed of MA, CA, NC, and p6; influenza virus polyprotein composed of hemagglutinin, nucleoprotein, and neuraminidase).

The terms “Pol” and “polymerase,” “Rep” and “replicase,” refer to various viral proteins involved in viral nucleic acid replication (e.g., immunodeficiency virus polyprotein composed of the protease, reverse transcriptase, RNaseH and integrase enzymes; influenza virus polyprotein composed of polymerases PB1, PB2, and PA; SARS virus polyprotein composed of Rep 1a, Rep 1b, and proteases).

The terms “Env” and “envelope,” “M” and “membrane,” “S” and “spike” refer to the virus surface proteins/polyproteins (e.g., immunodeficiency virus polyprotein gp160 composed of gp120 surface and gp41 transmembrane; coronavirus polyprotein composed of spike or S protein, membrane protein, and envelop protein).

As used herein, the term “regulatory protein” refers to the small viral proteins involved in modulation of the viral replicative cycle, including Tat, and Rev in the immunodeficiency virus.

As used herein, the term “accessory protein” refers to the small viral proteins whose functions have been shown to be dispensable inl vitro, including but not limited to Nef, Vpu, Vpr, and Vif in the immunodeficiency virus.

The tenn “suitable for” as used herein, refers to a condition or a combination adapted to a specific use or purpose. In some embodiments, “suitable for” refers to conditions for administration of a vaccine to a subject; as such this term encompasses but is not limited to an appropriate vaccine dosage (e.g., less than 10cc), an appropriate vaccine formulation (e.g., alum adjuvant), and an appropriate vaccine schedule. In other embodiments, “suitable for” refers to a particular combination of vectors/constructs suitable for production of a letumsome, or VLP.

As used herein, the term “immune response” refers to the alteration in the reactivity of an organism's immune system upon exposure to an antigen. The term “immune response” encompasses but is not limited to one or both of the following responses: antibody production (e.g., humoral immunity), and induction of cell-mediated immunity (e.g., cellular immunity including helper T cell and/or cytotoxic T cell responses).

The term “adult” refers to adolescent and mature subjects. The term “youth” refers to immature subjects (e.g., children). The term “neonate” refers to newborn subjects (e.g., babies).

The term “route” as used herein, refers to methods for administration of a prophylactic or therapeutic agent. In some embodiments, “route” refers to the method of administration of a vaccine including but not limited to intramuscular, intravenous, intraperitoneal, subcutaneous, oral, intranasal, intravaginal, intrarectal, and stomacheal administration methods.

As used herein, the term “physiologically acceptable solution” refers to an isotonic solution such as an aqueous solution comprising for example, saline, phosphate buffered saline, Hanks' solution, or Ringer's solution.

The term “infected” as used herein, refers to a subject in which a pathogen has established itself. In preferred embodiments, the term “infected subject” refers to a subject that is infected with a virus of interest. In contrast, the term “uninfected” refers to a subject in whom a pathogen has not established itself (but who may have been exposed to another pathogen). In preferred embodiments, the term “uninfected subject” refers to a subject that is not infected with a virus of interest. In the context of the invention, the term “uninfected subject” encompasses subjects whom are not infected with a virus of interest (e.g., SCV, HIV, etc.), but who may be infected with a different virus or viruses (e.g., CMV, EBV, etc.).

The term “control” refers to subjects or samples that provide a basis for comparison for experimental subjects or samples. For instance, the use of control subjects or samples permits determinations to be made regarding the efficacy of experimental procedures. In some embodiments, the term “control subject” refers to animals that receive a mock treatment (e.g., empty vector).

As used herein, the term “antibodies reactive with” refers to antibodies that bind to an antigen of interest. In preferred embodiments, the term “antibodies reactive with” is used in reference to antibodies that bind to a virus of interest (or to a viral protein).

The term “cytotoxic T lymphocytes reactive with” refers to cytotoxic T lymphocytes capable of lysing an MHC (e.g., HLA)-matched cell presenting epitopes derived from an antigen of interest. In preferred embodiments, the term “cytotoxic T lymphocytes reactive with” is used in reference to cytotoxic T lymphocytes or CTLs capable of lysing a MHC-matched cell infected by a virus of interest, or presenting epitopes derived from viral proteins.

The term “helper T lymphocytes reactive with” refers to helper T lymphocytes capable of secreting lymphokines in response to an MHC (e.g., HLA)-matched cell presenting epitopes derived from an antigen of interest. In preferred embodiments, the term “helper T lymphocytes reactive with” is used in reference to helper T lymphocytes or TH cells capable of secreting lymphokines in response to an MHC-matched cell infected by the virus of interest, or presenting epitopes derived from viral proteins.

As used herein, the term “induced an immune response” refers to an immune response elicited by a vaccine or a set of vaccines of the present invention.

The term “cloning” refers to the use of nucleic acid manipulation procedures to produce multiple copies of a single gene or gene fragment of interest.

As used herein, the term “mutating” refers to the use of one of a number of procedures (e.g., site-directed mutagenesis, chemical mutagenesis, etc.) for altering a nucleic acid sequence. In preferred embodiments, the term “mutating” refers to the use of molecular techniques for introducing deleterious changes to an immunodeficiency virus gene. Deleterious changes include but are not limited to premature stop codons in a viral gene and substitutions that destroy viral enzymatic activity.

The term “wild-type,” as used herein, refers to a gene or gene product having characteristics of a gene or gene product as isolated from a naturally occurring source. A wild-type gene is that which is most frequently observed in a population and is thus arbitrarily designed the “normal” or “wild-type” form of the gene.

In contrast, the term “mutant,” as used herein, refers to any changes made to a wild-type nucleotide sequence, either naturally or artificially. In preferred embodiments, the mutant nucleotide sequences encodes a translation product that functions with enhanced or decreased efficiency in at least one of a number of ways including, but not limited to, specificity for various interactive molecules, rate of reaction and half life. As used herein, the term “mutation” refers to a permanent transmissible change in genetic material encompassing but not limited to substitutions, deletions, and insertions.

As used herein, the term “ligating” refers to the process of joining nucleic acid fragments together.

The term “synthesizing” refers to processes of artificially manufacturing oligonucleotides or polypeptides.

As used herein, the term “classifying” refers to the categorization of vaccines into desired groups.

As used herein, the term “virulent” refers to markedly pathogenic viruses (e.g., viruses causing severe disease).

The term “distinct” refers to viruses that are distinguishable from one another (e.g., different strain, clade, host cell tropism, etc.).

As used herein, the terms “vector” and “construct” refer to tools used to transfer nucleic acid sequences from one cell to another or from one organism to another. Appropriate vectors for use with the methods and systems of the present invention include but are not limited to plasmids (e.g., pcDNA3.1), cosmids, and yeast artificial chromosomes. In other embodiments, the term “live vector” refers to recombinant bacteria, fungi, and viruses (e.g., vaccinia virus, adeno-associated virus, poliovirus, etc.).

The terms “expression vector,” “expression construct,” “promoter and terminator,” “expression cassette” and “plasmid,” as used herein refer to a recombinant nucleic acid molecule containing a desired coding sequence and appropriate regulatory sequences necessary for the expression of the operably linked coding sequence in a cell of interest. The nucleic acid molecule may be either double or single-stranded. Nucleic acid sequences necessary for expression in prokaryotes usually include a promoter, an operator (optional), and a ribosome-binding site (often along with other sequences). Eukaryotic cells are known to utilize promoters, enhancers, and termination and polyadenylation signals.

The terms “in operable combination,” and “operably linked” as used herein refer to the ligation of nucleic acid sequences in a manner suitable for directing transcription and/or translation of the nucleic acid sequences.

As used herein, the term “strain” refers to a group of presumed common ancestry, but with some clear-cut genetic distinctions (e.g., not clones). In preferred embodiments, the term “strain” is used in reference to distinct virus isolates.

The term “active site” refers to a specific region of an enzyme where a substrate binds (binding site) and catalysis (catalytic site) takes place.

As used herein, the term “viral enzyme” refers to viral proteins that catalyze chemical reactions of other substances without being destroyed or altered upon completion of the reactions. The terms “protease” and “Pro” refer to a viral enzyme that catalyses the splitting of interior peptide bonds in a protein. The terms “reverse transcriptase” and “RT” refer to a viral enzyme involved in the synthesis of double stranded DNA molecules from single stranded RNA templates. The terms “RNase H” and “Ribonuclease H” refer to a viral enzyme that specifically cleaves an RNA base paired to a complementary DNA strand. The terms “integrase” and “IN” refer to a viral enzyme that inserts a viral genome into a host chromosome.

The terms “hemagglutinin,” “HA” and “H” refer to influenza spike-form viral surface antigens having specific serologic reactivity. The terms “neuraminidase,” “NA” and “N” refer to spike-form viral surface antigens with specifically serological reactivities encoded by NA genome segment in influenza A virus. Similarly, the terms “NA” and “N” also refer to viral nucleocapsid proteins in other viruses such as HIV and coronavirus. The terms “polymerase,” “PB1,” “PB2,” and “PA” refer to a viral enzyme that incorporates nucleic acids into a gene or genome. Similarly, the terms “replicase,” “Rep 1a,” and “Rep 1b” also refer to a viral enzyme that incorporates nucleic acids into a gene or genome. The terms “nucleoprotein” and “NP” refer to a viral capsid proteins. The terms “membrane protein” and “M” refer to a viral protein that becomes incorporated into viral membrane. The terms “matrix,” “MA,” and “M” refer to a viral protein that interacts with the viral genome and plays a role in viral assembly. The terms “envelope protein,” “envelope” “Env” and “E” refer to a viral protein that becomes incorporated into the viral envelope. The terms “non-structural proteins” and “NS proteins” refer to viral proteins that are not necessarily incorporated into a viral particle (i.e. influenza A viral NS1 is expressed in cytoplasm but is not present in the virion, or NS2-NS3 proteinase in hepatitis C virus). The terms “core” and “C” refer to a viral nucleocapsid protein that makes up a viral particle.

As used herein, the terms “long terminal repeat” and “LTR” refer to homologous nucleic acid sequences several hundred nucleotides in length that are found at either end of a proviral DNA, and are formed by reverse transcription of retroviral RNA. LTRs are thought to play an essential role in integrating the provirus into the host DNA. In proviruses, the upstream LTR acts as a promoter and enhancer and the downstream LTR acts as a polyadenylation site.

The term “adjuvant” refers to a substance added to a vaccine to improve the immune response (e.g., alum). As used herein, the term “molecular adjuvant” refers to proteins that improve the immunogenicity of a vaccine or to the genes encoding these proteins. The term “molecular adjuvant” encompasses, but is not limited to costimulatory molecules, cytokines, chemokines, growth factors, etc.

As used herein, the term “costimulatory molecule” refers to a molecule on the surface of or secreted by an antigen presenting cell or APC that provides a stimulus or second signal required for activation of T cells. The term “costimulatory molecule” encompasses, but is not limited to B7-1 and B7-2 (CD80 and CD86).

The term “cytokine” refers to small proteins or biological factors (in the range of 5-20 kD) that are released by cells and that have specific effects on cell-cell interactions, communication and activity. The term cytokine encompasses, but is not limited to interleukins and lymphokines.

The term “chemokine” refers to cytokines that are chemotactic for leukocytes. They are subdivided into two groups on the basis of the arrangement of a pair of conserved cysteines. The CXC chemokines have paired cysteines separated by a different amino acid, and are chemoattractants for neutrophils but not monocytes. The CC chemokines have adjacent cysteines, and are chemoattractants for lymphocytes, monocytes, eosinophils, basophils, but not neutrophils. The term “chemokine” encompasses, but is not limited to platelet factor-4, platelet basic protein, interleukin-8, melanoma growth stimulatory protein, and macrophage inflammatory protein 2.

The term “growth factor” refers to biological factors that are produced by the body to control growth, division and maturation of cells. “Growth factors” include, but are not limited to epidermal growth factor, platelet-derived growth factor, fibroblast growth factor, etc.

The term “introducing” as used herein refers to the any suitable process for bringing foreign nucleic acid (e.g., DNA) into cells. Thus, the term “introducing” encompasses but is not limited to transfection, transformation, transduction, etc. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, biolistics (i.e., particle bombardment), and the like.

As used herein, the terms “polymerase chain reaction” and “PCR” refer to the method of K. B. Mullis described in U.S. Pat. Nos. 4,683,195, 4,683,202, and 4,965,188, hereby incorporated by reference. Briefly, PCR is a method for increasing the concentration of a segment of a target sequence in a DNA mixture without cloning or purification. Because the desired amplified segments of the target sequence become the predominant sequences (in terms of concentration) in the mixture, they are the to be “PCR amplified.” Similarly, the term “modified PCR” as used herein refers to amplification methods in which a RNA sequence is amplified from a DNA template in the presence of RNA polymerase or in which a DNA sequence is amplified from an RNA template the presence of reverse transcriptase.

### DESCRIPTION OF THE INVENTION

The present invention relates to methods and compositions for manufacturing virus-like particles (VLPs), suitable for use as immunogens and as research tools. The VLPs of the present invention provide a safe alternative to the use of pathogenic viruses for clinical and laboratory applications. In some embodiments, the VLPs are used as prophylactic vaccines, while in other embodiments the VLPs are used as therapeutic vaccines. The systems of the present invention are suitable for producing recombinant VLPs corresponding to either RNA or DNA viruses.

Pathogenic Viruses

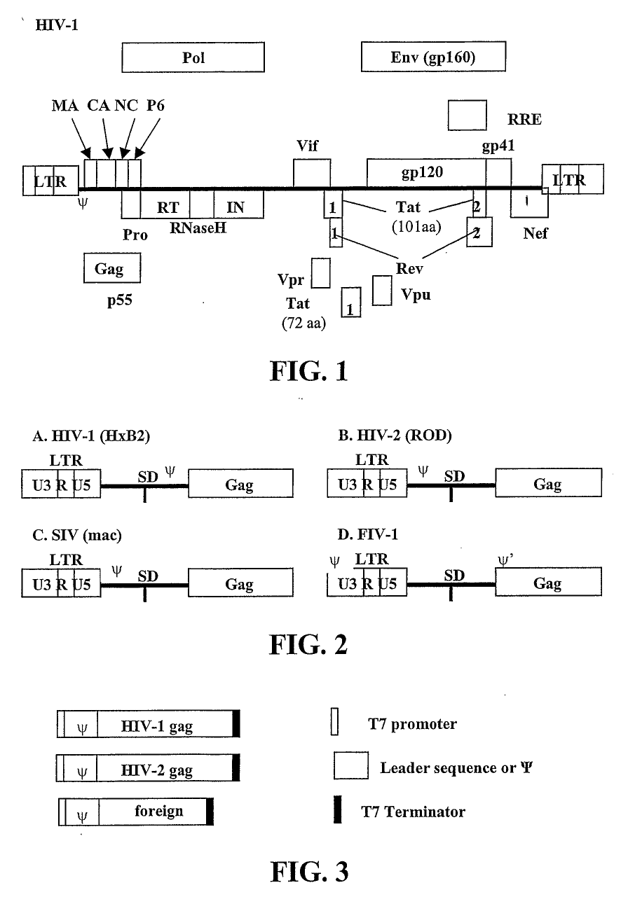

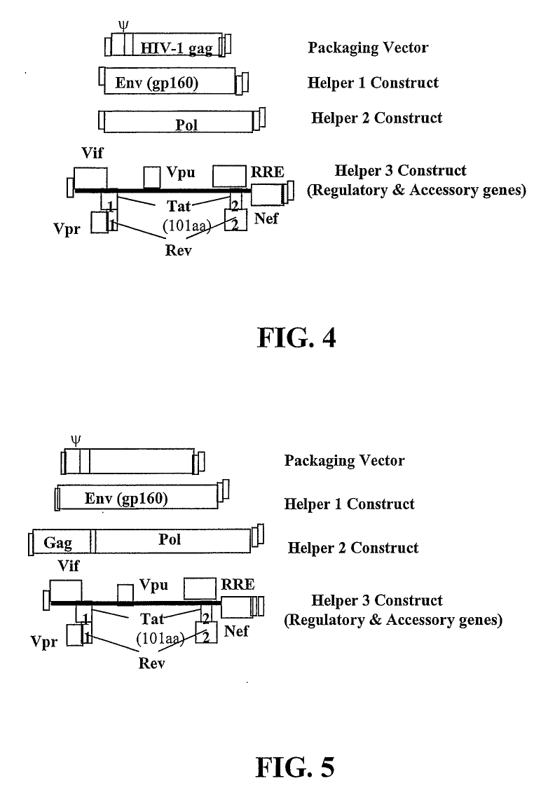

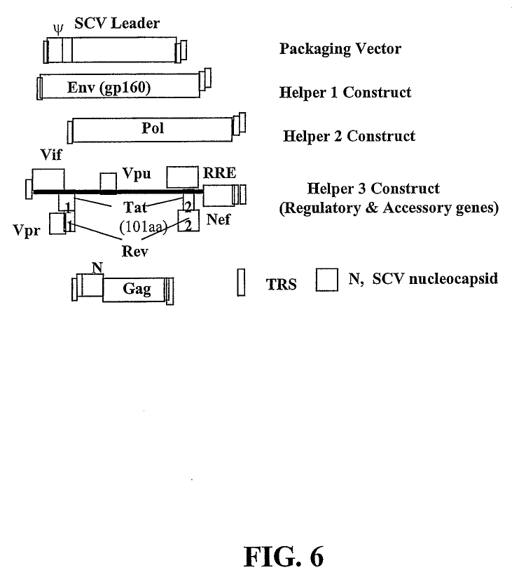

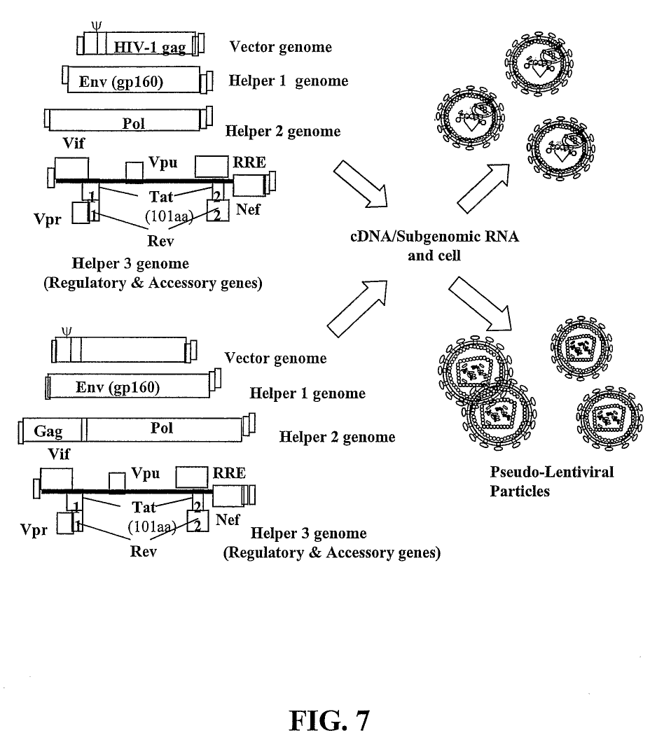

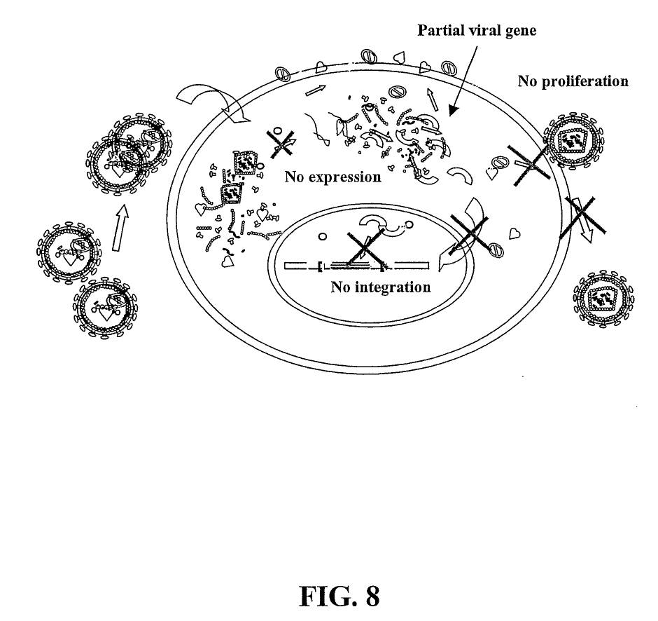

For example, the human immunodeficiency virus (HIV) is a single-stranded RNA virus of about 9.7 kilobases (Muesing et al., Nature, 313:450-458, 1985). The double stranded DNA form of HIV is known as the provirus. The HIV provirus can exist as an integrated linear form with both ends flanked by the long terminal repeats (LTRs) or as an unintegrated circular form with one or two LTRs (Gallo et al., Nature, 333:504,1988; Pang et al., Nature, 343:85-89, 1990; and Teo et al., J Virol, 71:2928-2933, 1997). The central genes of the proviral DNA encode more than nine proteins that are classified as major structural proteins (Gag, Pol, and Env); regulatory proteins (Tat and Rev); and accessory proteins (Vpu, Vpr, Vif, and Nef) (Emerman and Malim, Science, 280:1880-1884,1998). As seen in the schematic of the HIV-1 genome provided in FIG. 1, some of these proteins are cleaved into smaller functional proteins or enzymes during the process of viral maturation. For example, the 55-kilodalton (kD) Gag precursor protein is cleaved into four smaller proteins designated MA (matrix or p17), CA (capsid or p24), NC (nucleocapsid or p9), and p6; while 160 kD Env (gp160) is cleaved into gp41 and gp120 (Gottlinger et al., Proc Natl Acad Sci USA, 86:5781-5785, 1989).

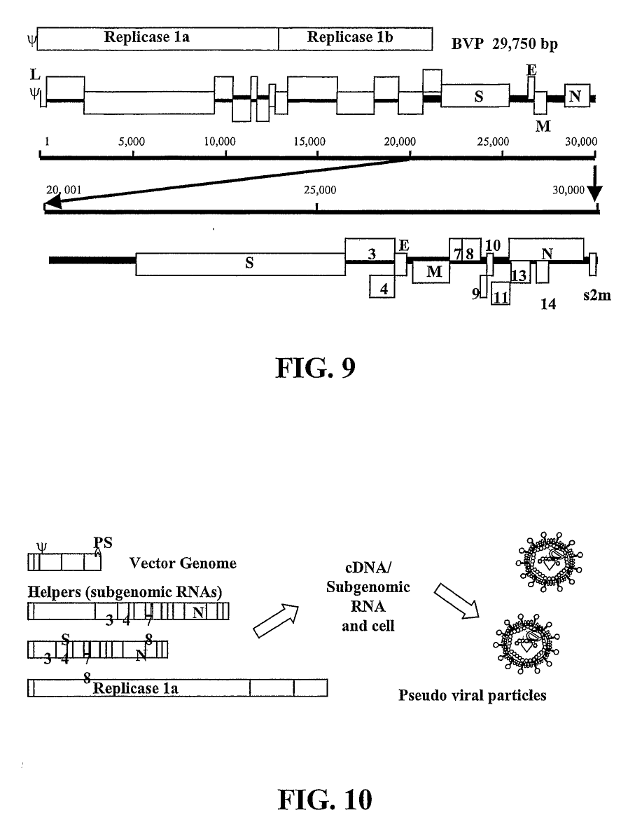

Severe acute respiratory syndrome (SARS) coronavirus (SCV) is a spherical virus of the coronavirus family containing a basic set of four essential structural proteins: membrane (M) protein, small envelope (E) protein, spike (S) glycoprotein, and nucleocapsid (N) protein, in addition to its polymerase/replicase (Pol/Rep 1a and Rep 1b) proteins (Enjuanes et al., Virus Taxonomy, Classification and Nomenclature of Viruses. Academic Press: New York, pp 827-849, 2000). These five major components are conserved in the coronavirus family members. The N protein wraps the genomic RNA into a nucleocapsid that is surrounded by a lipid membrane in which the S, M, and E proteins are found. The M and E proteins are essential and sufficient for viral envelope formation (Vennema et al., Virol, 181:327-335, 1991). The M protein also interacts with the N protein, presumably to mediate the assembly of the nucleocapsid into the virion (Narayanan and Makino, J Virol, 75:9059-9067, 2001). Trimers ofthe S protein form the characteristic spikes that protrude from the virion membrane. The S protein is responsible for viral attachment to specific host cell receptors, which is the basis for the narrow host range specificity of these viruses, and for cell-cell fusion (Cavanagh, The Coronaviridae. Plenum Press, Inc., New York, pp. 73-103, 1995). As seen in the schematic of the SCV genome provided in FIG. 9, the unique elements of the SCV are novel open reading frames (Orfs) between S and E and between M and N. The virus contains Orf 3, Orf 4, Orf 7, Orf 8, Orf 9, Orf 10, Orf 13 and Orf 14 (Rota et al., Science, 300:1394-9, 2003; and Marra et al., Science, 300:1399-404, 2003). The SCV genome is a capped, polyadenylated, nonsegmented, infectious, positive-strand RNA molecule of 29,750 bp. Its 5′ two-thirds is occupied by Orf la and Orf lb, from which the viral replication and transcription functions are derived. Downstream of Orf lb a number of genes are found that encodes the structural and several nonstructural proteins. These genes are expressed through a 3′-coterminal nested set of subgenomic mRNAs, synthesized by a process of discontinuous transcription. The subgenomic mRNAs represent variable lengths of the 3′ end of the viral genome, and each provides at its 5′ end a sequence identical to the genomic 5′ leader sequence (van der Most and Spaan, Coronavirus Replication, Transcription, and RNA Recombination, Plenum Press: London, pp. 11-31, 1995). The mRNAs are each functionally monocistronic and the proteins are translated only from the 5′-end of most of the Orfs.

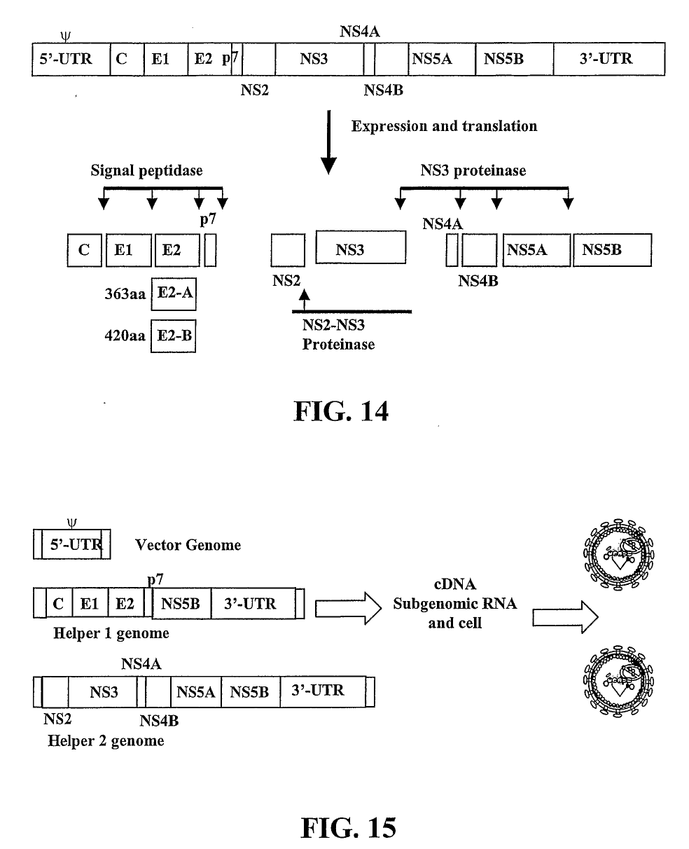

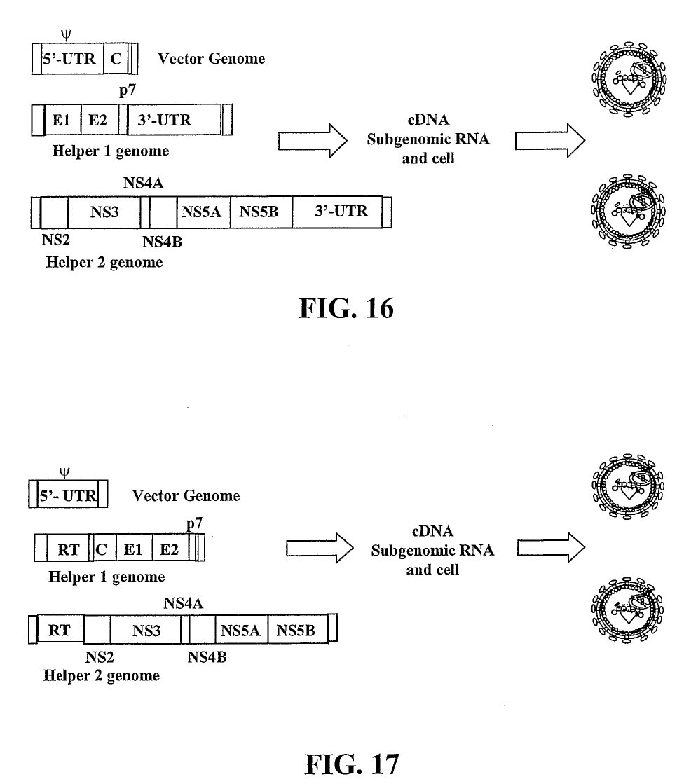

Hepatitis C virus (HCV) is a small, enveloped, positive-strand RNA virus belonging to the Flaviviridae family. Like HIV, the HCV genome is remarkably variable with more than 30 genome types and numerous quasispecies. As seen in the schematic of the HCV genome provided in FIG. 14, the HCV genome (approximately 9.6 kb) contains a single long open reading frame encoding a polyprotein precursor of approximately 3100 amino acids, translationally cleaved by both the host and viral proteases to yield at least 10 structural and nonstructural proteins in the order of N-tenninus-Core-Env (E1)-E2-p7-NS2 (NS2)-NS3-NS4A-NS4B-NS5A-NS5B-C-terminus (Gianini and Brechot, Cell Death Differ, 10(Suppl 1):S27-38, 2003). The core protein and the envelope proteins, El and E2 are structural proteins, followed by p7, a protein of unknown function. The six nonstructural viral proteins function in polyprotein proteolysis, polymerase activities, and the formation of a membrane-associated replicase complex (Kato, Acta Med Okayama, 55:133-59, 2001).

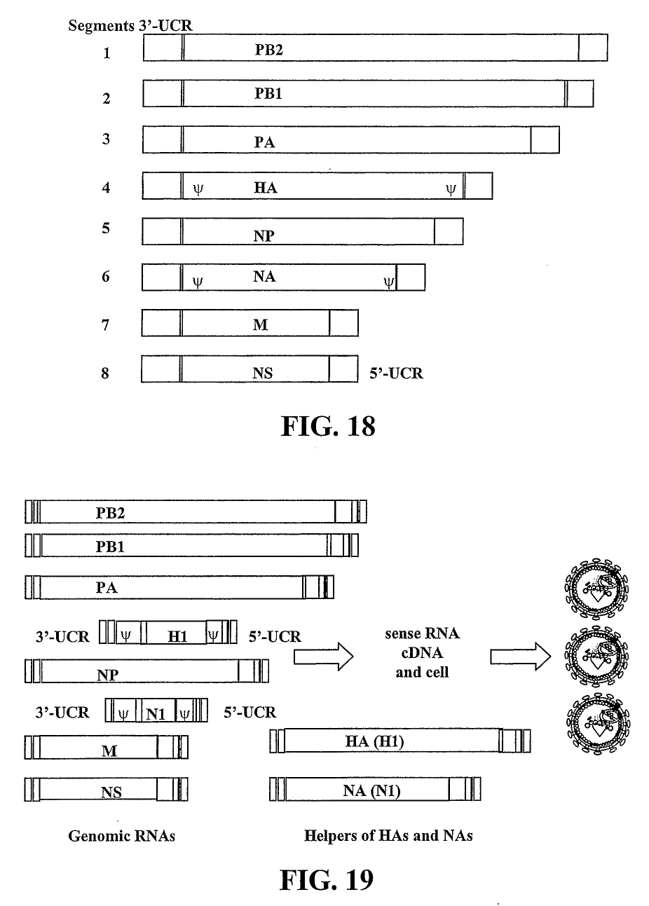

Influenza virus has eight single-stranded RNA genome segments of negative polarity, each encoding specific viral proteins as seen in the schematic of the ANIH1 genome provided in FIG. 18. Each genomic segment encodes one or two proteins termed hemagglutinin (HA or H), neuraminidase (NA or N), matrix (M1 and M2), nucleoprotein (capsid and/or NP), viral transcriptase complex (PB-2, PB-1 and PA), and non-structural proteins (NS, NS1 and NEP) (Lamb and Krug, Orthomyxoviruses, In: Knipe and Howley (eds.), Fields Virology, vol. 1, 4th ed., Philadelphia, pp. 1487-531, 2001; and Hilleman, Vaccine. 20:3068-87, 2002). Fifteen different H subtypes numbered 1 to 15, and nine N subtypes numbered 1 to 9, are used to specify the H and N formulae for strains of influenza A virus (Wright and Webster, Orthomyxoviruses, In: Knipe and Howley (eds.), Fields Virology, vol. 1, 4th ed., Philadelphia, pp. 1533-79, 2001; and Levin et al., Math Biosci, 188:17-28, 2004). Numbering of the subtypes of type A viruses is based on the immunologic specificities of their H and N proteins, even if each H or N is phenotypically different in other respects. For instance, the 1957 pandemic H2N2 virus was derived by reassortment of H1N1 with an unidentified virus possessing new H2, N2, PB1 and PA genes (Scholtissek et al., Virology, 87:13-20, 1978; and Kawaoka et al., J Virol, 63:4603-8, 1989).

Encapsidation Systems

The present invention provides multiple encapsidation systems suitable for producing a wide variety of VLPs, each containing only a defined portion of a viral genome. Importantly, the vaccines and vaccination regimens of the present invention are contemplated to elicit an immune response in immunized individuals, without risk of viral infection. Thus, the present invention is contemplated to prevent and treat infectious viral diseases in human subjects. The vaccines of the present invention are administered to subjects sequentially or in combination. Additionally, the disclosed methods are suitable for use in generating VLPs corresponding to viruses of nonhuman animals (e.g., bovine rotavirus, raccoon poxvirus, bat rabies virus, feline leukemia virus, canine distemper virus, fish rhabdovirus, and great ape Ebola virus). Thus, the vaccines and vaccine regimens of the present invention are also contemplated to prevent and treat infectious viral diseases in nonhuman animals.

Some embodiments of the present invention, provide methods for producing VLPs comprising: providing an artificial viral genome or packaging vector comprising a nucleic acid sequence having one or more specific packaging signal sequences, and two or more helper constructs or expression vectors comprising viral nucleic acid sequences. The nucleic acid sequences of the packaging vectors and the helper constructs are transcribed and translated in vivo (transfected cells) or in vitro (via transcription/translation systems), to form VLPs. An exemplary HIV VLP is produced from an HIV VLP encapsidation system comprising an artificial viral genome or packaging vector comprising a nucleic acid sequence having a specific packaging signal sequence and two or more expression vectors or helper constructs comprising one or more HIV open reading frames. In some preferred embodiments, the HIV nucleic acid sequences of the packaging vector or the helper construct comprise mutant HIV genes. Viral proteins encoded by the mutant HIV genes are enzymatically nonfunctional, but immunologically similar (B and T cell epitopes are conserved) to wild type HIV proteins. In an exemplary embodiment, an HIV encapsidation system comprises: a packaging vector comprising a nucleic acid sequence having a 5′ promoter, in operable combination with a packaging signal, a gag ORF, a terminator sequence, and a 3′ polyA sequence; a first helper construct comprising a nucleic acid sequence having an env and pol genes; and a second helper construct comprising a nucleic acid sequence having a vif-vpr-tat-rev-vpu-RRE-nef poly gene. The env and pol genes of the first helper construct, and the poly gene of the second helper construct are flanked by a 5′-promoter and 3′-terminator, respectively.

Testing of the VLP vaccines and vaccination regimens of the present invention is done in nonhuman primates or lower animals, and comprises: inoculating subjects with the VLP vaccines; and measuring VLP-specific immune responses elicited by the VLP vaccines. These steps are repeated (two or more times) until a pre-determined immune response is obtained. In some embodiments, the subjects are then challenged with virulent viral strains, to determine whether the VLP vaccines are suitable for eliciting protective or therapeutic immune responses.

In preferred embodiments, the VLP vaccines are administered to subjects by intramuscular (IM) injection and/or oronasal (ON) spray. However, other routes of inoculation, including but not limited to, intranasal (IN), oral (PO), intravaginal (IVG), intravenous (IV) and intrarectal (IR), are also suitable. hli further embodiments, purified viral proteins and/or cytokines are administered to boost the immune responses elicited by the VLP vaccines.

The present invention is discussed in more detail below, and is exemplified in the encapsidation systems described herein for HIV/SIV, SCV/FIPV, HCV, and influenza A viruses (H1N1, H5N1, H7N3), tested in the rhesus macaque model, the feline model, the chimpanzee model, and the chicken model, respectively. However, the present invention is not limited to these viruses, and/or animal models, which are provided merely as illustrations. Analogous procedures and techniques are equally applicable to other RNA viruses (e.g., HTLV, RSV, Japanese encephalitis virus, St Louis encephalitis virus, Murray Valley encephalitis virus, West Nile virus, Polio, food and mouth disease virus, Hanta, Nipah, influenza, Ebola, etc.), other DNA viruses (e.g., smallpox, HSV, HAV, HBV, HPV, HHV, CMV, EBV, adenovirus, poxvirus, iridovirus, etc.), and other subjects (e.g., African green monkeys, cattle, sheep, seals, fish, mice, pigs, humans, etc.). In addition, the encapsidation system of the present invention is suitable for use with other vectors (e.g., SFV, YFV 17D vector, AAV vector, BCG vector, pcDNA3.1 (±), pCEFL, etc.).

Selection of Viral Genes

The main barriers to the development of effective virus vaccines as exemplified by the failure of attenuated and inactivated viral vaccines are: inadequate virus attenuation or incomplete inactivation; extensive viral sequence variation; viral integration into the host genome leading to latent infection; and viral transmission by virions as well as by infected cells. In addition, some attenuated and inactivated vaccine viruses have reverted into more virulent forms, thereby causing disease in individuals with compromised immune systems. In addition, such vaccine viruses have been observed to cause birth defects, particularly if the vaccine is given in the first trimester of pregnancy. Some vaccine viruses that have reverted to a more virulent form or that have been inadequately inactivated spread more easily from vaccinated to unvaccinated individuals. Moreover, it is also possible for some live virus vaccine strains to recombine with wild type viral strains. To overcome these barriers to the generation of effective virus vaccines, the viral strains and viral genes used in the VLP vaccines of the present invention are carefully selected. In preferred embodiments, the sequences of an infectious viral isolate (e.g., SIV_{mac251}, HIV-1_{89.6}, HIV-2_{ROD}, SCV HK-39, etc.) or the consensus sequences of multiple infectious virus isolates (e.g., HIV-1 BVP, HIV-2 BVP, SCV BVP, HCV BVP, AH5N1 BVP, etc.) are used.

For illustration purposes, exemplary artificial viral genomes or packaging vectors are shown in the figures. Unless otherwise indicated, the packaging vector insert comprises a packaging signal (PS) and a viral or foreign nucleic acid, which is flanked by a 5′ promoter and a 3′ terminator and polyA sequence. Suitable immunodeficiency virus packaging vector inserts include but are not limited to nucleic acid fragments comprising: a PS-gag-pol fragment; a PS-gag fragment; and a PS- foreign gene fragment. In some embodiments the PS is an immunodeficiency virus PS, while in other embodiments the PS is a foreign (non-immunodeficiency virus) PS. Suitable inserts for immunodeficiency virus helper constructs include but are not limited to nucleic acid fragments comprising: an env fragment; a pol fragment; a gag-pol fragment; and regulatory/accessory protein gene fragments. However, under no circumstances do the helper construct inserts comprise LTR sequences or packaging signals. In the event that the packaging vector insert comprises a foreign PS, then one of the helper construct inserts comprise a foreign gene or fragment encoding a protein capable of recognizing the foreign PS. Some viral genes, such as HIV pol or HIV protease (Pro), reverse transcriptase (RT), and integrase (IN), are used in either wild type or mutated forms. The gene arrangement and gene order used in vaccine constructs of the present invention may differ from the specific embodiments disclosed herein. For instance, in one embodiment, an AIDS VLP encapsidation system comprises a single helper construct comprising tat, rev, nef, vif, vpr, and vpu genes in any order, while in another embodiment, it comprises a first helper construct comprising tat and rev genes, and a second helper construct comprising nef, vif, vpr, and vpu genes. Alternative embodiments comprise multiple helper constructs each comprising a single viral gene. In some embodiments, the HIV tat gene of the helper construct(s) comprises two tat genes, an early fully spliced tat gene and a late incompletely spliced tat gene. Further embodiments of the present invention comprise a helper cell line that constitutively or inducibly expresses one or more viral genes of interest (stable transfectants). In particularly preferred embodiments, the encapsidation system of the present invention comprises a nearly complete viral genome (e.g., all ORFs in the absence of the integration sequences of the viral LTRs). In some embodiments, the VLPs of the include gene products from distinct immunodeficiency viruses or strains (e.g., HIV-2 gag, pol, and/or env). Thus, the VLPs of the present invention comprise many of the desirable properties of an inactivated or attenuated virus without the safety considerations. In particular, the VLPs produced by using the encapsidation systems of the present invention are essentially “dead viruses” with respect to their inability to replicate.

For an AIDS VLP, the viral genes of the encapsidation system are derived from any immunodeficiency virus, including but not limited to primate viruses (e.g., HIV-1, HIV-2, SIV, SHIV) and nonhuman animal viruses such as BIV or JDV (cattle), FIV (cat), MVV or OLV (sheep), CAEV or OLV (goat), and EIAV (horse). If not specifically identified, HIV encompasses all subtypes of either HIV-1 or HIV-2. HIV-1 encompasses all subtypes of HWV-1. HIV-2 encompasses all subtypes of HIV-2, etc.

For a SARS VLP, the viral genes of the encapsidation system are derived from any coronavirus or torovirus of the Coronaviridae family, including but not limited to primate viruses (e.g., HCV 229E, HCV OC43, monkey SCV, HTV) and nonhuman animal viruses such as BCV and BTV (cattle), FIPV and FCV (cat), PRCV, TGEV, PEDV, PHEV and PTV (swine), IBV (birds), CCV (dog), and ETV (horse). If not specifically identified, SCV encompasses all subtypes of either human SCV or animal SCV-like viruses. HCoV encompasses all subtypes of human coronaviruses, etc.

For an HCV VLP, the viral genes of the encapsidation system are derived from any hepatitis C viruses, including various quasi-species. If not specifically identified, HCV encompasses all subtypes of HCV.

For an influenza VLP, the viral genes of the encapsidation system are derived from any influenza viruses, including but not limited to primate viruses (e.g., influenza A virus, influenza B virus, and influenza C virus) and nonhuman animal viruses such as bovine influenza A virus (cattle), porcine influenza A virus (swine), equine influenza A virus (horse), and marine influenza A and B viruses (marine mammals). If not specifically identified, influenza A virus encompasses all subtypes of influenza A viruses. Influenza B virus encompasses all subtypes of influenza B viruses. Influenza C virus encompasses all subtypes of influenza C viruses, etc.

Construction of Virus-Like Particles (VLPs)

The encapsidation system and VLPs of the present invention are not limited to any particular RNA or DNA virus. For illustration purpose only, four exemplary types of VLPs corresponding to various RNA viruses (e.g., HIV, SCV, HCV, and influenza A virus) are described in detail.

Exemplary AIDS VLPs are constructed from chemically synthesized proviral DNA or from proviral DNA that has been subdloned into a vector of interest. HIV/SIV DNA is either treated with restriction enzymes or PCR amplified to produce fragments of the desired size. Computer programs are utilized to assist in the design of the various HIV/SIV constructs comprising appropriate promoter sites, terminator sites, restriction enzyme sites, and/or PCR primer sites. Alternatively, restriction sites or PCR primer sites located on the multiple cloning site of an HIV/SIV vector are utilized for subdloning. If a particular sequence of interest lacks a desired restriction site, then specific oligo-primers are designed to generate viral DNA fragments comprising the sites of interest. Additionally, point and cassette mutations, start and stop codon insertions, gene fusions, gene recombinations and rearrangements, are employed as necessary to generate the desired viral gene fragments. The gene fragments are then size-fractionated and purified by for example, agarose gel electrophoresis. Purified fragments are ligated into appropriate expression vectors (e.g., plasmids). In some embodiments, the packaging vector and helper constructs are prepared within a single vector backbone, while in other embodiments different vector backbones are utilized. For instance, in one embodiment a vector/construct comprises a T7 promoter and a T7 terminator, while another vector/construct comprises a RNA polymerase II (pol II) promoter and polyA sequences. Other vector/constructs comprise a SP6 promoter and SP6 terminator or a CMV promoter.

The encapsidation system of the present invention comprises one or more expression vectors such as those derived from viruses or phages (e.g., CMV, T7 phage, and pol II, etc), or those derived from plasmids (e.g., pCR3.1, pcDNA3.1, pCEFL, pCAGGS, etc.). Well-established methods are used to construct recombinant AIDS VLPs comprising HIV/SIV genes in operable combination with promoters and other regulatory sequences to control expression of the genes (Ausubel et al., Current Protocols in Molecular Biology Greene Publishing Associates and Wiley biterscience: NY, 1989; and Sambrook et al., Molecular Cloning: A Laboratory Manual Cold Spring Harbor Laboratory: NY, 1989). In some preferred embodiments, a pCAGGS plasmid is used as an expression vector, and an HIV/SIV sequence is introduced into suitable cell lines capable of expressing the HIV/SIV genes. The pCAGGS plasmid comprises a CAG promoter comprising the cytomegalovirus (CMV) immediate-early enhancer, the chicken β-actin promoter, and the rabbit β-globin polyadenylation signal (Niwa et al., Gene, 108:193-200, 1991).

In some instances, a special initiation signal is used for efficient translation of inserted HWV/SIV sequences. Exogenous transcriptional control signals, including an ATG initiation codon, are provided in some embodiments. Furthermore, initiation codons are inserted in phase with the viral reading frame to ensure proper translation. These exogenous translational control signals and initiation codons are of either natural or synthetic origin. In addition, the efficiency of expression is enhanced in some embodiments by the inclusion of appropriate transcription enhancer elements, such as an additional promoter, transcription terminators, etc. (Bitterner et al., Methods in Enzymol, 153:516-544, 1987; and Sato et al., Science, 273:352-354, 1996).

The present invention also encompasses modifications of the virus fragments such as the incorporation of epitope or fluorescent protein tags, in order to more easily monitor expression of the HIV/SIV gene products. Some protease cleavage sites are introduced between genes in order to generate appropriately sized proteins. When used as vaccines, the VLPs are generally administered in an adjuvant or pharmaceutically acceptable diluent (the nature of which is influenced by the intended route of administration).

In some embodiments, the nucleic acids from which the VLPs are produced are first transcribed into mRNA species and then introduced into a packaging cell line to produce virus-like particles. In other embodiments, when the vectors/constructs comprise eukaryotic promoters such as CMV, the nucleic acids from which the VLPs are produced are transcribed in vivo in a transfected eukaryotic cell (e.g., Human PBMC, human PBL, MT-2 cells, H9, TALL-1, CCRF-CEM, MT-4, C8166, CEMx174, RC-10, CEM, CD4+ Hela cells, PT63, CEM-SS, Molt-4, HUT 78 cells, rhesus PBMC, PBL, HOS.CD4 cell lines, CrFK cells, feline PBMC, PBL, FL4 cells, FeT-J cells, MBM cells, MYA-1 cells, peripheral blood T cells, lymphocytes and lymphoid cell). In some embodiments, the VLPs are expressed from vectors/constructs comprising a selectable marker, thereby permitting the selective growth of stably transfected cells.

Similarly, SARS VLPs are constructed from completely chemically synthesized SCV DNA or reverse transcribed SCV DNA subcloned into a vector/construct of interest. In contrast to HIV/SIV, SCV has six subgenomic sequences in addition to a large genome, and SCV RNAs are synthesized via a viral RNA-dependant RNA polymerase (without a DNA intermediate). Subgenomic negative strands contain a complementary copy of the leader sequence at their 3′-ends to serve as the templates for synthesis of subgenomic mRNAs. Provirion transcription is signaled by transcription regulation sequences (TRSs). Therefore, more than six helper constructs are generally used to produce SCV VLPs. At least one TRS is inserted into the vectors/constructs between promoter and viral genes in order to transcribe SCV viral RNAs. Because the extent of the complementarity between the 3′-end of the SCV leader sequence and the sequences flanking the 3′-end of the TRS influences mRNA levels, the MRNA abundance is not directly proportional to the potential base pairing between the 3′-end of the viral genomic leader and the sequence complementary to TRS (Alonso et al., J Virol, 76:1293-308, 2002). Two TRS sequences or extended TRS are therefore introduced into each clone. Preferred cell lines for producing SCV VLPs include but are not limited to monkey Vero E6 cells and feline Felis catus whole fetus (FCWF) cells.

HCV VLPs are also constructed from completely chemically synthesized DNA or from reverse transcribed HCV DNA subdloned into a vector(s) of interest. Preferred cell lines for producing HCV VLPs include but are not limited to B cell lymphoma cell lines.

Influenza A VLPs are constructed from completely chemically synthesized cDNA or from influenza A cDNA subcloned into a vector(s) of interest. In contrast to HIV/SIV, influenza A virus is a minus-strand RNA virus having eight separate segments. For this reason, a special vector system is used to express both positive and negative strand RNAs. Two artificial genomes or packaging vectors are constructed, one comprising a NA fragment and the second comprising an HA fragment. The remaining six gene segments are cloned into a suitable vector backbone and simultaneously used as both packaging vectors and helper constructs. Two additional helper constructs for HA and NA are also prepared. The RNA polymerase I (pol I) promoter and terminator sequences flanked by a RNA polymerase II (pol II) promoter and polyA sequences are introduced. This complete pol I-pol II transcription unit is flanked on one side by an RNA polymerase II (pol II) promoter and on the other by a polyadenylation site. The orientation of the two transcription units of this pol I-pol II system allows the synthesis of negative strand (anti-sense) viral RNA and positive strand (sense) mRNA from one viral cDNA template (Hoffinann et al., Proc Natl Acad Sci USA, 97:6108-13, 2000). Human and animal cell lines including but not limited to Madin-Darby canine kidney (MDCK) cells, Vero cells, 293T human embryonic kidney cells, and COS-7 cells are used for the production of influenza A VLPs.

Vaccination Regimens

The VLP vaccines of the present invention are contemplated to be suitable for both prophylactic and therapeutic applications. In preferred embodiments, the VLP vaccines elicit VLP-reactive humoral and cellular immune responses.

Single VLP Method

Briefly, a virus infects a cell through the binding of its viral surface protein(s) (e.g., gp120 of HIV, spike protein of SCV, Env E1/E2 of HCV, and HA of influenza A virus, etc.) to one or more host cell receptor(s) (e.g., CD4 for HrVs, ACE2 for SCV, CD81 as a co-receptor for HCV, sialic acid receptor for influenza A viruses, etc). After binding, the viral and cellular membranes fuse, leading to entry of the virus core particle into the host cell cytoplasm. DNA viruses are generally transported to juxtanuclear locations where they synthesize mRNA and undergo a second uncoating (Hunter, In Fields Virology, 4th ed., Philadelphia, pp. 171-197, 2001). Poxvirus DNA replication and protein translation takes place in the cytoplasm, followed by packaging and release of new virions from the infected cell (Moss, In Fields Virology, 4th ed., Philadelphia. pp.2849-2883, 2001). Most RNA viruses that lack a DNA phase replicate in the cytoplasm. However, several negative-stranded RNA viruses, such as influenza, Thogoto, and Boma disease viruses, take advantage of the host cell's nuclear machinery for replication of their RNA genomes and for synthesis of their mRNAs. The newly synthesized RNA is translocated into the cytoplasm for translation, and packaged as virions for subsequent release from the host cell (Cros and Palese, Virus Res, 95:3-12, 2003). For RNA viruses having a DNA phase (e.g., HIV), viral RNA is reverse transcribed by the viral RT as a provirion and integrated into the host cell genome. Transcription of the provirion is regulated by transcription factors such as Sp1 and the TATA-binding factors, members of the NF-KB family, and an early form of tat. A full-length viral RNA is produced and spliced into short viral mRNAs and exported to the cytoplasm for translation. Increased Rev concentrations lead to the export of unspliced and singly spliced viral RNA to the cytoplasm for translation of late viral proteins and RNA packaging into the viral particle. The viral gag and gag-pol proteins are cleaved by the viral protease to generate mature virions. At a later stage in the infection cycle, HIV-1 particles are packaged at cell surface with the viral Env protein. Thus certain steps in the life cycle of most viruses are shared, namely attachment to host cell surface receptors, entry into the cell, uncoating of the viral nucleic acid, and replication of the viral genome. After new copies of viral proteins and genes are synthesized, these components spontaneously encapsidate into progeny virions, which then exit the cell.

A novel approach to the production of VLPs has been taken is described herein. Briefly, a DNA of interest is cloned into three or more plasmids that serve as templates for the in vitro synthesis of recombinant viral DNA/RNA. Encapsidation systems comprising two helper constructs have a reduced likelihood of producing replication-proficient viruses through RNA recombination (e.g., Smerdou and Liljestrom, J Virol, 73:1092-1098, 1999), and thus yield safe VLPs. The DNA/RNA is subsequently transfected into human or animal cells by electroporation or chemical transfection. The expression system includes an in vivo packaging process employing two or more packaging-deficient helper constructs and a packaging vector to form VLPs. The VLPs of the present invention lack multiple viral genes, as only a partial viral genome or an artificial genome is packaged. For example, RNA replication of HIV VLPs is cytoplasmic, and integrase activity is aborted, so there is little risk of viral integration into the host genome. In some embodiments, the enzymatic activity of RT is abolished by mutation to further increase the biosafety of the system. In one embodiment, the HIV VLPs contain a complete set of viral proteins, but lack HIV env, regulatory and accessory genes, as only the gap-pol gene of the packaging vector contains the necessary packaging signal. In another embodiment, the pol gene is not attached to the gag gene, so that the nascent VLPs contain only the gag gene. This expression system can also be used in an in vivo packaging process through direct infection of host cells, by employing a suitable promoter (e.g., the cytomegalovirus immediate early promoter).

The viral VLPs can “infect” cells through the binding of the VLP surface antigens to host cell receptors and coreceptors. After binding, the VLPs gain entry into the host cell through membrane fusion or endocytosis, and disassembly subsequently occurs within the host cell cytoplasm. However, in the absence of a certain viral genes (e.g., those included in the helper constructs), initiation of expression of a complete set of viral gene products does not occur (e.g., regulatory protein Rev and accessory protein Vpu of HIV, TRS sequence and N protein and M protein of SCV, C and E1/E2 protein of HCV, HA and NA of influenza A virus, etc.). Thus, although the VLPs of the present invention are able to initiate an abortive infectious process, they are unable to replicate due to a failure in an early step in the process (prior to encapsidation, and budding/release of virions). The use of these VLPs as vaccines is contemplated to be suitable for induction of potent VLP-reactive immune responses, without the risk of infecting unvaccinated recipients and/or disease development (Seligman and Gould, Lancet, 363:2073-5, 2004).

The VLP vaccines used in the common VLP method include a VLP vaccine containing one subtype or multiple subtype VLPs from one kind of viruses (e.g., for AIDS, from HIV-1_{89.6} (clade B) or HIV-1_{89.6}, HIV-192RW016 (clade A), HIV-1 93IN101 (clade C), HIV-1 93UG070 (clade D), etc). For a VLP vaccine that is used to elicit an immune response to a particular quasispecies, say HIV-1 BVP (clade A), only one VLP vaccine is used. For a VLP vaccine that is used to elicit an immune response to multiple subtypes, a VLP cocktail containing HIV-1 BVP subtypes B is used.

Multiple Homogeneous VLP Method

It has previously been shown, that HIV-2 infection does not result in protection against subsequent HIV-1 infections (van Der Loeffet al., AIDS, 15:2303-10, 2001). However, it is contemplated that infection with HIV-2 is not equivalent to vaccination with HIV-2 genes or gene products. Similarly, influenza A viruses are known to escape from host immunity through antigenic alterations arising through genetic drift (principally by point mutations of the HA gene), or through genetic shift (reassortment of HA genes). Currently, partial control of influenza spread is attained by using inactivated, subunit, or live virus vaccines, all of which rely on worldwide surveillance to predict the immunologic specificity of the next influenza A virus (Hilleman, Vaccine, 20:3068-87, 2002; and Hay et al., Philos Trans R Soc Lond B Biol Sci, 356:1861-70, 2001). Thus, for protection against more than one influenza virus, vaccines containing more than one VLP are utilized. For AIDS, a VLP cocktail comprising VLPs of HIV-1 92RW016 (clade A), HUV-1_{89.6} (clade B), HIV-1 93IN101 (clade C), HIV-1 93UG070 (dlade D) and/or further HIV clades is contemplated to protect the immunized subjects from infection with many strains of HIV-1. In some embodiments, HIV-2 genes or epitopes are administered to subjects as additional VLPs (e.g., HIV-2_{ROD} and HIV-2 BVP, etc) when it is desirable to elicit a broadly reactive immune response. It is contemplated that once an immune response against the gene products of both HIV-1 and HIV-2 VLPs (including a majority of the HIV-1 and HIV-2 proteins) have been elicited, that the vaccinated subjects are protected from HIV-1 and HIV-2 infection and/or disease onset/progression (e.g., AIDS). Similarly, a cocktail of homologous VLPs comprising FIV-subtypes A, B and C is contemplated to protect immunized cats from FIV infection and/or feline AIDS caused by FIV as evidenced by a dual-subtype inactivated FIV vaccine (Uhl et al., Inmunol Immunopathol. 90:113-32, 2002). Similarly, a cocktail of influenza A virus VLPs comprising H1N1, H3N2, H5N1, H7N7, and H9N2 is contemplated to protect the subjects from influenza A virus infection. Further inclusion of influenza B VLP(s) and influenza C VLP(s) is contemplated to protect subjects from multiple influenza viruses. It is contemplated that once an immune response against the viral gene products of influenza A, influenza B, and influenza C VLPs (including a majority of the influenza A, influenza B, and influenza C proteins) have been elicited, that the vaccinated subjects are protected from influenza infections and/or disease onset (e.g., fever, respiratory symptoms, and lymphopenia). Similarly, a cocktail of HCV quasispecies VLPs is contemplated to protect vaccinated subjects from HCV infection and/or disease progression (e.g., hepatitis and hepatocellular carcinoma).

Thus, the multiple VLP vaccines used in the multiple homogeneous VLP method encompasses the vaccines previously described for the common VLP methods, in addition to gene products from other virus strains (e.g., HIV-1_{89.6}, influenza A H7N3, etc.) or from related viruses (e.g., HIV-2, influenza B, and influenza C, etc.). The use of VLP vaccines containing viral proteins from other strains of virus is contemplated to enhance the immune response of the inoculated subjects. For AIDS, this method encompasses the administration of one or more VLP vaccines comprising both HIV-1 and HIV-2 proteins to a subject under conditions suitable for inducing an immune response against the VLP vaccines.

The VLP vaccines used in the multiple homogenous VLP method include multiple VLPs of a specific virus (e.g., HIV, or influenza A, or SCV, or HCV, etc). A VLP cocktail comprising HIV-1 BVP subtypes A, B, C, and D is expected to elicit broadly reactive immune responses (e.g., responses against HIV infections not specifically included in the VLP cocktail such as HIV-1 subtypes E, F, G, H, I, J, K, N, and O). Similarly, a cocktail of H1N1, H3N2, H5N1, H7N7, and H9N2 influenza A VLP vaccine is expected to elicit a broadly reactive immune response.

Multiple Heterogeneous VLP Method

The introduction of multiple live attenuated vaccines, such as the combined measles-mumps-rubella (MMR) vaccine or the combined multivalent measles-mumps-rubella-varicella (MMRV) vaccine, has dramatically reduced the occurrence of those diseases. A combined multivalent vaccine also offers the convenience of a single injection and thus facilitates implementation in routine immunization schedules (Hesley et al., Pediatr Infect Dis J, 23:240-5, 2004). However, serious adverse events, such as high fevers, and measles-like rashes, have been reported. It is also been reported that attenuated vaccines with more than one virus have enhanced pathogenicity profiles (Woods et al., J Vet Diagn nvest, 11:400-7, 1999). Recent development of a tetra-combined DNA vaccine (Bacillus anthracis, Ebola, Marburg, and Venezuelan equine encephalitis virus) has been shown to elicit protective immunity in guinea pigs (Riemenschneider et al., Vaccine. 21:4071-80, 2003). However, such vaccines have not been proven to be safe in humans. In addition, DNA vaccines are not free of complications and safety concerns (Giri et al., Clin Microbiol Rev, 17:370-89, 2004; Stephenson, Expert Rev Vaccines, 1:355-62, 2002; and Griffiths, Rev Med Virol, 13:69-70, 2003).

The present invention further provides multiple heterologous VLPs that are contemplated to be suitable for preventing and treating a number of viral diseases. A heterologous VLP cocktail comprising HIV, HTLV, and HCV VLPs is contemplated to protect the vaccinated subjects from infections and diseases caused by HIV, HTLV, and HCV. Similarly, a heterologous VLP cocktail comprising SCV, influenza A virus, and RSV VLPs is contemplated to protect vaccinated subjects from infections and diseases caused by SCV, influenza A virus, and RSV. Similarly, a heterologous VLP cocktail comprising torovirus, coronavirus, picobimavirus, and pestivirus VLPs, is contemplated to protect vaccinated subjects from diarrhea and gastroenteritis caused by toroviruses, coronaviruses, picobimaviruses, and pestiviruses. It is contemplated that once an immune response against the viral gene products of the heterologous VLP cocktails has been elicited that the vaccinated subjects are protected from infections and/or disease progression (e.g., AIDS, SARS, and hepatitis). In some embodiments, the VLPs used in the multiple heterogeneous VLP method comprise many different VLPs (e.g., HIV, influenza A, SCV, and HCV, etc). For instance, a VLP cocktail comprising five heterologous VLPs is contemplated to elicit an immune response against all five VLPs.

Administration

For preventive purposes, VLP vaccines are administrated in an increased and then decreased protocol. Subjects are first given a set of VLP vaccine(s) either oronasally (IO) or intramuscularly (IM) in a single immunization (IX dose) on day one. The first inoculation is followed by two-booster immunizations intraoronasally with 2-5X VLP dose 2 days after the first immunization, and 10-30X VLP dose 6 days after the first immunization. After the first three inoculations (administered during week one), the subjects are immunized twice at 2-week intervals. Wild type virus is administered to each control and experimental subject at 4 weeks and 3 months following completion of the vaccine regimen. After each vaccination, enzyme-linked immunosorbent assays are performed to detect host antibody responses or vaccine antigen production. At the same time neopterin, beta2-microglobulin, antibody, cytokine, cytotoxic T lymphocyte (CTL), CD4/CD8 ratios, and lymphocyte proliferation are assessed to analyze humoral and cellular Th1 and Th2 immune responses (Shacklett et al., J Virol, 76:11365-78, 2002; Letvin et al., J Virol, 78:7490-7, 2004; Logvinoff et al., Proc Natl Acad Sci USA, 101:10149-54, 2004; Woo et al., J Clin Microbiol, 42:2306-9, 2004; de Haan et al., Virology, 296:177-89, 2002; de Jong et al., Dev Biol (Basel), 115:63-73, 2003; Ali et al., Clin Infect Dis, 38:760-2, 2004; Mooij et al., J Virol,78:3333-42, 2004; Deml et al., Methods Mol Med, 94:133-57, 2004; Nascimbeni et al., J Virol, 77:4781-93, 2003; Jiao et al., J Gen Virol, 85:1545-53, 2004; Zhu et al., Immunol Lett, 92:237-43, 2004; Glansbeek et al., J Gen Virol, 83:1-10, 2002; Chen et al., Virus Res, 103:147-53, 2004; and Babiuk et al., J Med Virol, 72:138-42, 2004.).

For systemic administration, the preferred routes of injection include but not limited to: intramuscular, oronasal, intravenous, intraperitoneal, and subcutaneous injections. The route utilized is dependent upon the vaccine formulation. Other preferred routes of administration include: oral, intranasal, intravaginal, intrarectal, and stomacheal. The vaccines of the present invention are formulated in solution, preferably in physiologically compatible buffers such as Hanks' solution, Ringer's solution, saline and phosphate buffered saline, or as adjuvants, or packaged in liposomes, or creams or other time-release agents. Alternatively, the vaccines of the present invention are formulated in a gas form, and absorbed or suspended in a physiologically acceptable solution under pressure for mucosal spray. In some embodiments, the vaccines of the present invention are formulated in a solid or lyophilized form, and dissolved or suspended in a physiologically acceptable solution immediately prior to use. Viral proteins of each vaccine group, and specific cytokines or chemokines are administered to subjects mounting low immune responses to the viral gene products of that group.

In situations in which subjects have previously developed nonspecific immune activation markers, circulating CD8+ T-cells are used as a vaccine-related activation marker for the cellular immune system. The magnitude of the humoral immune response is determined by measuring neutralizing antibodies. The same assays described above are used to detect the immune responses elicited by the VLP vaccines of first challenge. The necessity of the boosts is determined from the assays used to measure the VLP-reactive humoral and cellular immune responses.

To determine whether the nonhuman subjects have developed protective immune responses against a virus, a vaccine is administrated to experimental animals. In a preferred embodiment, high titers of neutralizing antibodies are detected and significant changes in activation markers (e.g., neopterin and beta2-microglobulin) are observed. A lethal strain of virus is administrated to the vaccinated subjects that have developed measurable immune responses against the VLP vaccine(s) (See, e.g., U.S. patent application Ser. No. 10/207,346). In particularly preferred embodiments, the vaccinated experimental animals are protected from infection with the challenge virus. For example, a lethal strain of HWV/SIV (Reinhardt et al., J Med Virol, 56:159-167, 1998; and Baba et al., Nat. Med. 5:194-203, 1999) is administrated to subjects that have developed measurable immune responses against the HIV/SIV VLPs. In particularly preferred embodiments, the vaccinated experimental animals are protected from HIV infection.

For therapeutic purposes, VLP vaccines are administrated in an equivalent amount protocol. Subjects are given a set of VLP vaccine(s) either oronasally (IO) or intramuscularly (IN) in a lX dose on day one. The subjects are immunized at one-week intervals until their viral load is undetectable. The subjects are then immunized with the same dose of VLP vaccines once a month for 6 months. After that, they are immunized once every three-months for life. In a preferred embodiment, when an HIV-infected human has been vaccinated, once the vaccination protocol has been performed, the subject develops an immune response and does not develop outward signs and symptoms of AIDS.

Animal Models

The VLP vaccines of the present invention are tested in an animal model (one of the virus natural nonhuman hosts) before administration to human subjects. For example, the SIV/HIV VLP vaccines are tested in a rhesus macaque model (Macaca mulatta). However, other suitable animal models include but are not limited to nonhuman primates such as chimpanzees, baboons, and marmosets, as well as lower animals such as cows, cats, rabbits, ferrets, swine, sheep, goats, chicken, seals, fish, and rodents. In a preferred embodiment, SIV is used with the rhesus macaque model. However, this rhesus monkey model is also suitable for testing SHIV, HIV-1, HIV-2, FIV, and BIV VLPs. Similarly in preferred embodiment, SCV VLPs are tested in the rhesus macaque, ferret, and cynomolgus macaque models. Furthermore in some embodiments influenza A VLPs are tested in chickens, and swine.

### EXPERIMENTAL

The following examples are provided in order to demonstrate and further illustrate certain preferred embodiments and aspects of the present invention and are not to be construed as limiting the scope thereof In the experimental disclosure which follows, the following abbreviations apply: eq (equivalents); M (Molar); μM (micromolar); mol (moles); mmol (millimoles); μmol (micromoles); umol (nanomoles); g (grams); mg (milligrams); μg (micrograms); ng (nanograms); l (liters); ml (milliliters); μl (microliters); cm (centimeters); mm (millimeters); μm (micrometers); nm (nanometers); ° C. (degrees Centigrade); U (units), mU (milliunits); min. (minutes); sec. (seconds); % (percent); kb (kilobase); bp (base pair); PCR (polymerase chain reaction), TCID₅₀ (50% tissue culture infectious dose); MID₅₀ (50% mucosal infectious doses); HIV (human immunodeficiency virus); SIV (simian inmuunodeficiency virus); SCV (SARS coronavirus); HCV (hepatitis C virus); CMV (cytomegalovirus); UTS (untranslated sequence); LTR (long terminal repeat); UTR (untranslated region); L (leader sequence); TRS (transcription regulation sequences); and gpp (gag-pol precusor).

Materials described in the following examples are available from: NIH (National Institutes of Health, AIfDS Research and Reference Reagent Program); Invitrogen (Invitrogen, Carlsbad, Calif.); CDC (Center of Disease Control, Atlanta, Ga.); BVP (Bowling Vaccine & Pharmaceutical Inc.; San Francisco, Calif.); and ATCC (American Type Culture Collection, Manassas, Va.).

### EXAMPLE 1

### HIV and SIV Strain and Viral Gene Selection

In an exemplary approach, bases 1-10277 of SIVmac251/HUT 78 (GENBANK Accession Number M19499; and Franchini et al., Nature, 328:539-543, 1987) are used as a DNA template. Target genes are synthesized biochemically or are amplified by PCR using a series of specific primers: gag (bases 1014-2561), pol (bases 2216-5386), gpp (gap-pol precursor; bases1014-5386), vif (bases 5316-5960), vpx-vpr (bases 5788-6420), tat (bases 6278-6573 and 8785-8884), rev (bases 6504-6573 and 8585-9041), untranslated sequences (UTS; bases 807-1013 and 6574-8784), env (bases 6580-9225), nef (bases 9059-9802), 5′-LTR (long terminal repeat; bases 1-806), and 3′-LTR (bases 9803-10277). Other genes are also subdloned including: matrix (MA; bases 1136-1446), capsid (CA; bases 1447-2303), nucleocapsid (NC; bases 2304-2450), and gag p6 (bases 2451-2561), viral protease (Pro; bases 2531-2827), integrase (IN; bases 4459-5175), gpp RNAse H (bases 2828-4015), env gp120 (bases 7006-8127) and env gp41 (bases 8125-9225), vpx (bases 5788-6126), vpr (bases 6127-6420), regulatory and accessory gene fragment (RAF; bases 5316-6126 and 6574-9802). Other immunodeficiency virus genes are amplified and subdloned in a similar manner. For instance, genes from SIV_{mac239} (GENBANK Accession Number M33262; and Rigier and Desrosiers, AIDS Res Hum Retro, 6:1221-32, 1990) and from HIV-2_{89.6} (GENBANK Accession Number U39362; and Collman et al., J Virol, 66:7517-7521, 1992) are cloned into vectors of the encapsidation system of the present invention, and transfected into a suitable cell line, resulting in production of primate immunodeficiency virus pseudovirions. In the same way, various HIV-2 sequences are also subdloned.

### EXAMPLE 2

### Coronavirus Strain and Viral Gene Selection

In another exemplary approach, bases 1-29751 of SCV Tor2 (GENBANK Accession Number AY274119; and Marra et al., 300:1399-404, 2003) are used as a DNA template. Target genes are synthesized biochemically or are amplified by PCR using a series of specific primers: Leader sequence (bases 1-649), putative “packaging signal” (bases 19920-20225), replicase lab (bases 265-13392,13392-21485), spike glycoprotein (bases 21492-25259), Orf3 (bases 25268-26092), Orf4 (bases 25689-26153), small envelope E protein (bases 26117-26347), membrane glycoprotein M (bases 26398-27063), Orf7 (bases 27074-27265), Orf8 (bases 27273-27641), Orf9 (bases 27638-27772), Orf10 (bases 27779-27898), Orf11 (bases 27864-28118), nucleocapsid protein N (bases 28120-29388), Orf13 (bases 28130-28426), and Orf14 (bases 28583-28795). Other genes are also subdloned including: replicase 1a (bases 265-13392), replicase 1b (bases 13392-21485), A (bases 21492-28795), B (bases 25268-28795), C (bases 26398-28795), D (bases 27273-28795), and E (bases 27779-28795). Other SCV genes are amplified and subdloned in a similar manner. For instance, genes from SCV Urbani (GENBANK Accession Number AY278741; Rota et al., Science, 300:1394-9, 2003) and from HCV HK-39 (GENBANK Accession Number AY278491; and Zeng et al., Exp Biol Med, 228:866-73, 2003) are cloned into vectors of the encapsidation system of the present invention, and transfected into a suitable cell line, resulting in production of coronavirus pseudovirions.

### EXAMPLE 3

### Influenza Virus Strain and Viral Gene Selection

In another exemplary approach, bases 1-29751 of Influenza virus type A (H1N1) are used as a cDNA template. Target genes are synthesized biochemically or are amplified by PCR using a series of specific primers (in positive sense orientation): PB1 (bases 1-2341) (GENBANK Accession Number AF342823), PB2 (bases 1-2326) (GENBANK Accession Number M55469; and Schultz et al., Virology, 183:61-73, 1991), PA (bases 1-2233), (GENBANK Accession Number X17336; and Odagiri and Tobita, Nucleic Acids Res, 18:654, 1990.), HA (bases 1-1773) and mutated HA (mH1, bases 115-1727), (GENBANK Accession Number AF222026; and Olsen et al., Arch Virol, 145:1399-1419, 2000), NP (bases 1-1512), (GENBANK Accession Number AF342819), NA (bases 1-1458) and mutated NA (mN1, bases 205-1273), (GENBANK Accession Number AF342820), M (bases 1-1027), (GENBANK Accession Number M63519; Ito et al., J Virol, 65:5491-5498, 1991), NS (bases 1-890), (GENBANK Accession Number U53410), HA vector genome (truncated HA or tH1, bases 1-114 and 1727-1773), and NA vector genome (truncated NA or tN1, bases 1-204 and 1274-1458) Other influenza A virus genes are obtained and subcloned in a similar manner. For instance, HA gene from H1N1 Nanchang (GENBANK Accession Number AY180460; and Liu et al., Virology, 305:267-275, 2003) and NA gene from H1N1 Puerto Rico (GENBANK Accession Number NC004523; and Schickli et al., Philos Trans R Soc Lond, B, Biol Sci, 356:1965-1973, 2001) are cloned into vectors of the encapsidation system of the present invention, and transfected into a suitable cell line, resulting in production of influenza A virus VLPs. In the same way, various influenza A sequences (H5N1, H3N2, H7N7, H9N2) are also subcloned.

### EXAMPLE 4

### HIV Virus-Like Particle (VLP) Production

To construct HIV VLPs of the present invention, human immunodeficiency virus genes are manipulated through mutation, recombination, ligation, elongation, tagging, and deletion, for the purpose of generating recombinant single genes or multigenes that do not exist in the native HIV genome. For some genes, a eukaryotic start codon (e.g., ATG) and/or a stop codon (e.g., TAG, TAA, or TGA) is introduced. In some embodiments, the CMV immediate early promoter, the SP6 promoter or the T7 promoter, pol I promoter, pol II promoter, mouse hydroxymethylglutaryl-coenzyme A reductase (HMG) promoter, or adenovirus type 2 major late promoter (and/or enhancers), as well as a T7 terminator, SP6 terminator, pol I terminator, murine terminator sequences, hepatitis delta virus genomic ribozyme, or bovine growth hormone (BGH) polyadenylation site, are introduced into the encapsidation system packaging vectors and/or helper constructs. Additionally, a protease gene, such as the foot and mouth disease virus 2A autoprotease gene, and/or a protease cleavage site is introduced to permit the synthesis of processed proteins if required.

The active sites of four enzyme genes from pol (Pro, RT, RNAse H, and IN) are mutated with specific nucleotides in order to produce nonfunctional proteins with conserved three-dimensional structures. In some embodiments, cytokine genes, including but not limited to, IL-2, IL-12, and IL-15, Flt3 ligand, are also introduced into the packaging vector to enhance adaptive immune responses generated against the virus-like particle immunogens. The immunodeficiency virus genes described in Example 1 are cloned into the pCAGGS cloning vector (Niwa et al., Gene, 108:193-200, 1991) under control of regulatory sequences comprising the cytomegalovirus (CMV) immediate-early enhancer, the chicken P-actin promoter, and the rabbit β-globin polyadenylation signal. The pCAGGS expression vector permits the high level expression of recombinant genes in a wide variety of mammalian cell lines. In additional embodiments, other plasmids and vectors are used including but not limited to pKCB-Z (Sato et al., Science, 273:352-354, 1996), the pCEFL plasmid, and the pcDNA3.1 plasmid (Invitrogen). Various SIV clones are already available to expedite the subdloning process including for example pGEX-KGvpr (Wang et al., Virol, 211:102, 1995), pCMV-rev (Lewis et al., J. Virol. 64:1690, 1990), pTatC6H-1 (Purvis et al., AIDS Res Hum Retroviruses, 11:443, 1995). The identity of all constructs is confirmed by DNA sequencing.

To make an HIV VLP, several vectors are utilized. The first one is the packaging vector or vector replicon, which contains a minimum of a promoter, a packaging signal, and a terminator or polyadenylation site, thereby providing the initial signals required for viral encapsidation. In a preferred embodiment, a structural gene (e.g., gag, core, ribonucleoprotein, or nucleocapsid, etc.) is inserted after the packaging signal sequence or within the packaging signal sequence. Other (helper) constructs containing most if not all of the remaining viral genes are then generated. The genes from the helper constructs are expressed but since the helper constructs lack packaging signals, these genes are not assembled or packaged within the VLPs. Thus, the encapsidation system of the present invention is suitable for generating virus-like particles that lack a functional viral genome. For example, an HIV encapsidation system comprises a packaging vector, which in this case contains a packaging signal, and gag genes encoding MA, CA, and NC. The NC gag protein recognizes specific cis-acting RNA packaging signals (Strappe, J Gen Virol, 84: 2423-2430, 2003). Two helpers are also generated. The first helper comprises the replication signals and a subgenomic promoter in operable combination with genes encoding the HIV regulatory and accessory proteins, tat, vpr, vpu, vif, rev, and nef. The second helper comprises the genes encoding the HIV pol proteins PR, RT, and IN and env proteins gp120 and gp41. RNA is transcribed in vitro from the SP6 or T7 promoters of the helper plasmids. Alternatively, if the helper plasmids comprise eukaryotic promoters, RNA is transcribed in vivo (in transfected cells). A defined ratio of the three vectors is transfected into cells. The RT encoded by the first vector amplifies all of the viral RNA species. However, only the gag containing RNA is packaged into viral particles because the sequences required for HIV RNA packaging are localized to motifs in NC. Other RNAs produced from helper vectors lack this packaging signal and thus are not incorporated into the viral cores. Importantly, the HIV LTR is not contained in either the packaging vector or the helper constructs. Moreover, in preferred embodiments, inactivating mutations are introduced into the IN gene to prevent viral integration into the host cell genome. Additionally, if desired, the activity of the RT gene is also abolished by mutation. The use of three independent vectors also reduces the possibility of recombination among the viral RNA species thus avoiding formation of replication-competent viruses. Introduction of the three RNA species into specific cell types results in the formation of HIV VLPs, containing all the viral structural proteins, and the unique HIV regulatory and accessory proteins, in the absence of a complete HIV genome. When the HIV virus-like particle is administered to a subject, only a few proteins encoded by the gag genes are transcribed, and thus the VLPs cannot reproduce a complete set of viral proteins (e.g., rev, tat, and env protein). Furthermore, because the necessary regulatory and envelope proteins are lacking, new viral particles (progeny) are not produced. Enhancers, promoters, protease cleavage sites, and/or stop codons are introduced as needed into the helper vectors in order to achieve the desired expression pattern. These helper constructs are modified in various ways. For example, the cytomegalovirus (CMV) immediate early promoter is utilized in some embodiments to control expression of the HIV genes. In other embodiments, the hepatitis delta virus genomic ribozyme is utilized to ensure that a precise 3′-end of the viral RNA is obtained (Fodor et al., J Virol. 73:9679-82, 1999). In a preferred embodiment, a high level of transcription of the HIV genes of the helper constructs is obtained by using the CMV promoter to drive their expression.

The strategy outlined above is also suitable for the production of additional virus-like particles. For instance, the encapsidation system of the present invention is suitable for producing VLPs selected from but not limited to the following types: coronavirus, hepatitis virus, influenza virus, rabies virus, vesicular stomatitis virus, respiratory syncytial virus, Sendai virus, parainfluenza virus, rinderpest virus, Newcastle disease virus, bunyavirus, Hanta, Handra, West Nile virus, and Ebola virus.

### EXAMPLE 5

### SCV Virus-Like Particle Production

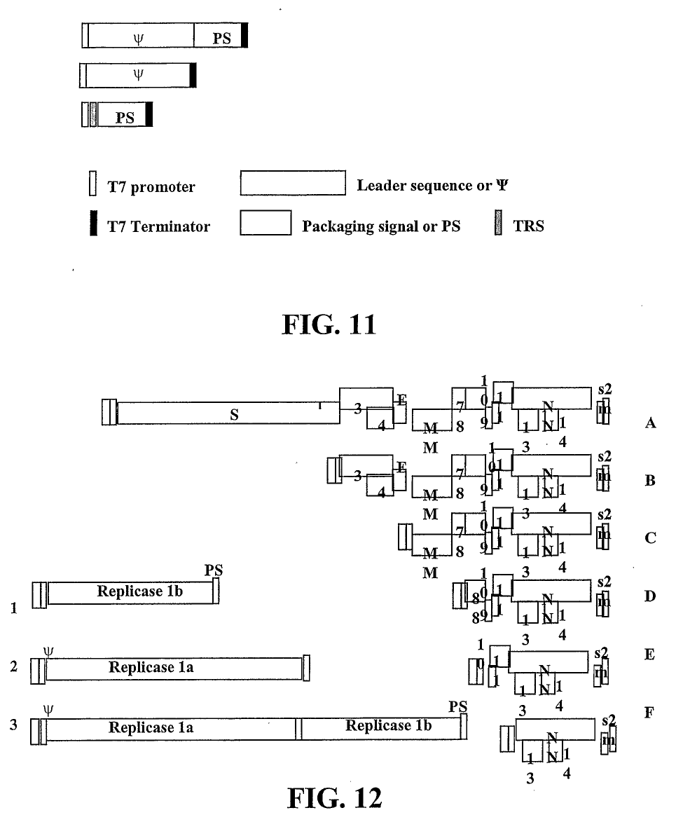

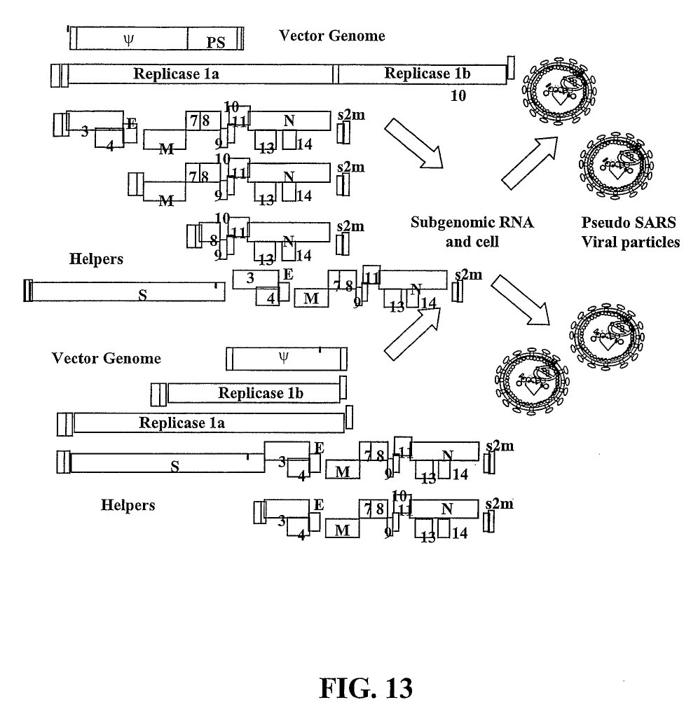

For illustrative purposes because the nature of SCV is different from that of HIV, a special gene or promoter is inserted into a SCV vector genome. Restriction sites for NarI, NotI, SacI, SfiI, SmaI, XmaI are not found in the SCV BVP sequence. PacI, NcoI, and SmaI restriction enzymes do not cut the nucleic acid fragment containing the leader sequence (bases 1-649) and the putative packaging signal sequence (bases19920-20225) of SCV of the BVP clone. As shown in FIG. 11, three packaging vectors based on the pIVEX2.4 (Roche) backbone are produced: 1) PLP, PacI-T7 promoter-Leader sequence (1-649)-packaging signal (19920-20225)-T7 terminator-polyA-PacI; 2) NL, NcoI-T7 promoter-Leader sequence (1-649)-polyA-T7 terminator-NcoI; and 3) SP, SmaI-T7 promoter-TRS-packaging signal (19920-20225)-polyA-T7 terminator-SmaI. All sequences are synthesized and cloned into pIVEX using the corresponding restriction enzymes. The final products are termed pPLP, pNL, and pSP.

Six putative SCV mRNAs with strong TRSs have been reported (Rota et al., Science, 300:1394-9, 2003; Marra et al., Science, 300:1399-404, 2003). In order to imitate the natural viral replication system while eliminating the possibility of non-specific encapsidation of viral mRNAs, a series of helper constructs are used for in vitro transcription and translation. FIG. 12 illustrates one embodiment of this approach. One panel of helpers (1-3, on the left) is constructed from replicase 1a and 1b. Each subgenomic helper has a T7 promoter, a TRS, a polyA sequence, and a T7 terminator added into their 5′- and 3 ′-ends, respectively. No leader sequence or packaging signal is included in the replicase helper constructs. If a given helper construct is not transcribed, a different TRS or more than one TRS is added. The second panel of helpers (A-F, on the right) is developed for the remaining SCV genes. TRSs for each construct differ in this embodiment: A helper comprises CAACTAAACGAACATG (SEQ ID NO:1); B helper comprises CACATAAACGAACTTATG (SEQ ID NO:2); C helper comprises GGTCTAAACGAACTAACT(40nt)ATG (SEQ ID NO:3); D helper comprises TCCATAAAACGAACATG (SEQ ID NO:4); E helper comprises AGTCTAAACGAACATG (SEQ ID NO:5); and F helper comprises TAAATAAACGAACAAATTAAAATG (SEQ ID NO:6). The sequence variations among different TRSs are contemplated to contribute to the varying strength of expression of the viral genes. For the constructs derived from replicase 1a and 1b genes, the same TRS identified from the leader sequence is utilized, TCTCTAAACGAACTTTAAAATCTGTGATG (SEQ ID NO:7). The minimal TRSs are underlined. If the viral protein expression is insufficient, two TRSs or an extended or modified TRS is introduced.

### EXAMPLE 6

### Influenza a Virus-Like Particle Production

Unlike HIV and SCV, which have a single positive strand RNA genome, influenza A virus has negative strand RNA genome composed of eight separate segments (See, e.g., FIG. 18; Capua and Alexander, Acta Trop, 83:1-6, 2002; Mikulasova et al., Acta Virol, 44:273-82, 2000; and Steinhauer and Skehel, Annu Rev Genet, 36:305-32. 2002). The principal antigens are the hemagglutinen (HA) and the neuraminadase (NA) glycoproteins of the virus envelope. HA is the predominant immunogenic component. The antigenic shift and drift among the 15 known HAs and the 9 known NAs results in the production of new variant or mutation strains that evade immunity of the host (Hilleman, Vaccine, 20:3068-87, 2002; and Stephenson and Zambon, Occup Med (Lond), 52:241-7, 2002). For this reason, an encapsidation system differing from than described for HIV and SCV is employed.

In a preferred embodiment, the pHW cloning vector is used as a backbone for ten plasmids each comprising a cDNA segment of the virus A/WS/33 (H1N1): PB2, PB1, PA, NP, M, NS, truncated vector segments tH and tN, and helper constructs comprising HA and NA (for background see for instance the eight plasmid system of Hoffinann et al., Proc Natl Acad Sci USA, 97:6108-13, 2000). pHW contains the pol I-pol II transcription system for synthesis of vRNA and mRNA. The cDNA of each of the eight influenza virus segments is inserted between the pol I promoter and the pol I terminator. This pol I transcription unit is flanked by a truncated immediate-early promoter (the pol II promoter) of the human CMV and by the BGH polyadenylation signal of the gene encoding bovine growth hormone. Two types of molecules are synthesized after transfection of the eight expression plasmids. From the human pol I promoter, negative strand vRNA is synthesized by cellular pol I. The vRNAs have NCRs at the 5′ and 3′ ends. Transcription by pol II yields mRNAs with 5′ cap structures and 3′ poly(A) tails. These mRNAs are translated into viral proteins. The ATG of the viral cDNA is the first ATG downstream of the pol II transcription start site. To ensure that the viral cDNAs of the expression plasmids, derived from RT-PCR amplification products, do not have unwanted mutations, the inserted cDNAs are sequenced.

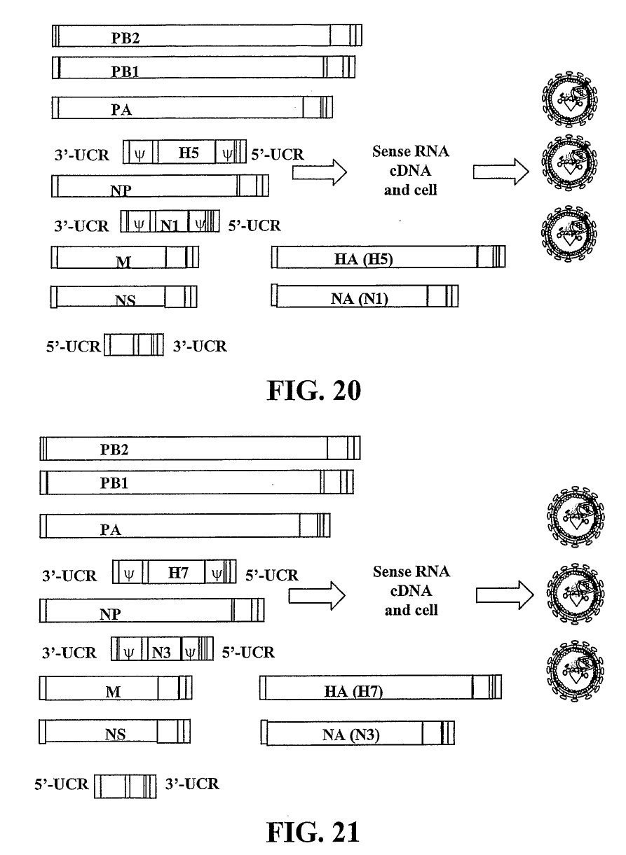

As shown in FIGS. 19-21, a preferred encapsidation system for production of influenza A VLPs comprises an HA packaging vector and an NA packaging vector, an HA helper construct and a NA helper construct, as well as helper constructs comprising the remaining influenza virus gene segments. The truncated HA and NA segments (tH1 and tN1, tH5 and tN1, and tH7 and tN3) comprise the only packaging sequences of the encapsidation system (Fujii et al., Tanpakushitsu Kakusan Koso, 48:1357-63, 2003; and Fujii and Kawaoka, Uirusu, 52:203-6, 2002), and thus are the only nucleic acids contained with the influenza virus-like particles. In contrast, both the HA and NA segments of the helper constructs are mutant HA and NA segments (mH1 and mN1, mH5 and mN1, and mH7 and mN3) lacking packaging sequences. Thus, introduction of the ten cDNA vectors/constructs into suitable cell types, results in the formation of an influenza VLP comprising the viral structural proteins (including the unique HA and NA glycoproteins) in the absence of genome segments corresponding to HA and NA. When the influenza virus-like particles are administered to a subject, the HA or NA glycoproteins encoded by the mutant HA and NA segments are not transcribed, and thus progeny influenza VLPs are not produced.

It has been reported that PB2, PB1, and PA proteins form a polymerase complex for transcription, and are associated at one end of each gene segment (Lamb and Krug, Fields Virology, vol. 1,. 4th ed, pp. 1487-531, 2001). Using a baculovirus expression system, others have isolated the PA-PB1-PB2 ternary complex termed 3P (Honda et al., Proc Natl Acad Sci USA, 99:13166-71, 2002). The 3P complex is assembled in a linear fashion (e.g., PA(C terminus)-(N terminus) PB1(C terminus)-(N terminus) PB2). The N-terminal region of PA and the C-terminal region of PB2 are not involved in the protein-protein contacts required for RNA polymerase assembly (Toyoda et al., J Gen Virol, 77:2149-57, 1996). In some embodiments, a plasmid containing 3P is used to express the influenza RNA polymerase for transcription and replication of the influenza A virus nucleic acids. This reduces the number of plasmids in the influenza A virus encapsidation system to eight. This approach is contemplated to eliminate any recombination between the nucleic acid sequence of the influenza virus-like particle and that of any co-infecting wild type influenza virus.

The strategy outlined for the influenza A virus, also applies to the production of virus-like particles for other influenza viruses including but not limited to influenza B viruses (ten plasmid system), influenza C viruses (nine plasmid system) and Thogotovirus (eight plasmid system).

### EXAMPLE 7

### Virus-Like Particle (VLP) Immunogens

In this example, sets I-III are homologous VLP immunogens of a single subtype, sets IV-VI are homologous VLP inununogens with more than one subtype, and sets VII-IX are heterogous VLP immunogens of more than two viruses. At least nine sets of VLP immunogens are utilized, a first set comprising an HIV VLP, a second comprising a SCV VLP, a third set comprising an influenza VLP, a fourth set comprising a cocktail of HIV-1 and HIV-2 VLPs, a fifth set comprising a cocktail of influenza A VLPs, a sixth set comprising a cocktail of SCV and human coronavirus VLPs, a seventh set comprising a cocktail of SCV, RSV, and influenza VLPs, an eighth set comprising a cocktail of HIV, HCV, and HTLV VLPs, and a ninth set comprising a cocktail of West Nile virus, LCM virus, Hanta virus, Dengue virus, Ebola virus, and Variola virus VLPs.

| | |
|---|---|
| Set I: | HIV-1 clade C, BVP strain and/or HIV-1 93IN101 VLPs |
| Set II: | SCV BVP and/or SCV HK-39 VLPs |
| Set III: | Influenza A virus H1N1 A/WS/33, and/or A/mallard/ |
| | Alberta/211/98, and/or A/Beijing/262/95, VLPs |
| Set IV: | HIV-1 clades A, B, C, D or more, and HIV-2 VLPs |
| Set V: | SCV BVP, SCV 229E, and SCV OC43 VLPs |
| Set VI: | Influenza A virus H1N1, H3N2, H5N1, H7N7, and |
| | H9N2 VLPs |
| Set VII: | SCV, RSV, and influenza VLPs |
| Set VIII: | HIV-1, HIV-2, HCV, and HTLV-I, and HTLV-II VLPs |
| Set IX: | West Nile virus, LCM virus, Hanta virus, Dengue |
| | virus, Ebola virus, and Variola virus VLPs |

Other virus strains suitable for use with the disclosed methods include, but are not limited to RNA viruses such as HIV, HCV, SIV, HTLV, RSV, SCV, Japanese encephalitis virus, St Louis encephalitis virus, Murray Valley encephalitis virus, rabies virus, West Nile virus, Polio, food and mouth disease virus, Hanta, vesicular stomatitis virus, Sendai virus, parainfluenza virus, rinderpest virus, Newcastle disease virus, bunyavirus, Nipah, and Ebola. In other preferred embodiments, the virus suitable for use with the disclosed methods include but are not limited to DNA viruses such as smallpox, HSV, HAV, HBV, HPV, HHV, CMV, EBV, adenovirus, poxvirus, simian virus, and iridovirus. Regrouping the VLPs is within the scope of the present invention, as is the alteration of the VLP order and number within each set.

### EXAMPLE 8

### Subjects

All experimental animals undergo a period of quarantine before initiation of the vaccine schedule. Each subject is subjected to pathology, parasitology, and bacteriology tests, and a complete health assessment including serological profile. In some embodiments, there is a preferred host or animal model in which VLP vaccine trials are conducted (e.g., chickens for influenza A virus, chimpanzee for HCV, ferret for SCV, fruit bat for Nipah, cat for FIPV, horse for EAIV, mouse for MHV, etc.). Thus, each animal model requires specific care and handling protocols. In a preferred embodiment of the present invention, each subject is screened for the specific pathogen of interest (e.g., by testing antibody reactivity to specific viruses). For preventive vaccine tests, healthy animals are utilized that are seronegative for the virus of interest. For therapeutic vaccine tests, infected animals are utilized that are seropositive for the virus of interest.

For illustrative purpose for AIDS vaccine research, animals are subjected to the following conditions. All experimental nonhuman primates undergo 2-week to 6-week quarantine before initiation of the vaccine schedule. Each subject is given three intradennal tuberculin tests, and hematology and serum chemistry profiles are taken. Additionally, a rectal swab is examined for bacterial cultures, feces are examined for occult blood, and ovum and parasite determinations are made. Importantly, the serum of each nonhuman primate subject is screened for antibody reactivity to: SIV, simian type D retrovirus, simian T-cell lymphotropic virus type 1, herpes B virus, and measles virus.

Specific-pathogen-free (SPF) cats are tested negative for toxoplasma, feline leukemia virus, and FIV before experimental infection. Blood, bone marrow, and lymph node tissue samples are collected from the jugular veins, the proximal end of femurs, and the popliteal lymph node, respectively for virological and immunological analyses. Cats are anesthetized for bone marrow aspiration and tissue collection and as needed for blood sampling.

New Zealand White rabbits are tested negative for Encephalitozoon cuniculi, Treponema cuniculi, Clostridium piliforme, Myxomatosis, RHDV, Toxoplasma sp., and CAR Bacillus. All rabbits are also screened for respiratory and enteric bacteria and for both ectoparasites and endoparasites before vaccine or antigen challenges.

Similarly, all human volunteers are given intradermal tuberculin tests and hematology and serum chemistry profiles are taken. Additionally, a rectal swab is examined for bacterial cultures, feces are examined for occult blood, and ovum and parasite determinations are made. Each human subject is also screened for antibody reactivity to: HIV-1, HIV-2, hepatitis A virus, hepatitis B virus, hepatitis C virus, Kaposi sarcoma-associated herpesvirus, human herpesvirus, cytomegalovirus, human papillomavirus, yeast and fungal infections.

### EXAMPLE 9

### Preventive VLP Vaccination Schedule

In some preferred embodiments, vaccines are administered oronasally (IO). However, other routes of vaccine administration (e.g., IM, IV, etc.) find use with the methods and compositions of the present invention. For illustrative purposes, four examples of the present invention are described below.

AIDS VLP Vaccine Trial

The AIDS vaccination schedule summarized here is for prophylactic use in HIV/SIV-seronegative subjects (uninfected). At the start of the experiments, the monkeys are inoculated IM/IO with 0.3 μg SIV VLP (as calculated by p27 contents). The first inoculation is followed by two-boost immunizations 10 with 1.0 [tg SIV VLP on day 2, then 10 μg SIV VLP on day 6. After three inoculations, the subjects are immunized twice with 0.3 μg SIV VLP at 2-week intervals, respectively. Immune responses (VLP-reactive T_{H}, CTL, Ab, and cytokine secretion) are measured. Wild type SIV_{mac239}, SIV_{mac251} or SHIV 89.6P are administered to each control and experimental subject at 4 weeks and 3 months following completion of the vaccine regimen. Specifically, four weeks after the last vaccination, half of the monkeys are challenged oronasally with a dosage of 10⁵ TCID₅₀. Three month after the last vaccination, the remaining monkeys are challenged oronasally with a dosage of 10⁵ TCID₅₀. Each vaccine group (e.g., VLP and/or VLP cocktail) or placebos are given at 0, 2, 6, 20, and 34-day intervals, to 10-20 neonatal subjects (1-3 months), 10-20 young subjects (4 month-5 years), and/or 10-20 adult adolescent and adult subjects (6 years and older).

SARS VLP Vaccine Trial

Similarly, the SARS vaccination schedule summarized here is for prophylactic use in SCV/FIPV-seronegative subjects (uninfected). An FIPV/SCV VLP or VLP cocktail (e.g., Set II and Set V) is utilized. At the start of the experiments, the kittens are inoculated IM/IO with 0.1 μg FIPV VLP (as calculated by N protein content). The first inoculation is followed by two-booster immunizations 10 with 0.3 jg FIPV VLP on day 2, then 1 μg after FIPV VLP on day 6. After three inoculations, the subjects are immunized twice with 0.1 μg FIPV VLP at 2-week intervals. Immune responses (VLP-reactive T_{H}, CTL, Ab, and cytokine secretion) are measured. Wild type FIPV 79-1146 is administered to each control and experimental subject at 4 weeks following completion of the vaccine regimen. Four weeks after the final immunization, all kittens are challenged oronasally with 1000 TCID₅₀ of FIPV 79-1146 (Haijema et al., J Virol, 77:4528-38, 2003). Each vaccine group (e.g., VLP and/or VLP cocktail) or placebos are given at 0, 2, 6, 20, and 34-day intervals, to 10-20 young (20 weeks) subjects.

Influenza VLP Vaccine Trial

Similarly, the influenza A vaccination schedule summarized here is for prophylactic use in influenza A virus HA type-seronegative subjects (uninfected). An influenza A VLP or VLP cocktail (e.g., Set III and Set VI) is utilized. At the start of the experiments, the SPF white leghorn chickens (Spafas) are inoculated subcutaneously at the base of the neck in one regular immunization with an amount of 50 ng AH5N1 VLP (as calculated by HA content). The first inoculation is followed by two-boost immunizations intranasally with 150 ng influenza A VLP on day 6, then 1.0 μg influenza A VLP on day 20. Immune responses (VLP-reactive T_{H}, CTL, Ab, and cytokine secretion) are measured. Wild type A/PR/8/34 (HlNl), A/GS/HK!437-4/99 (H5N1), A/Duck/Germany/1215/73 (H2N3), A/CK/HK/86.3/02 (H5N1), or A/CK/Hidalgo/28159-232/94 (H5N2) is administered to each control and experimental subject 3 weeks following completion of the vaccine regimen. Three weeks after the third immunization, all chickens are challenged intranasally with a dosage of 10 CLD₅₀ or 100 CLD₅₀ (Liu et al., Virology, 314:580-90, 2003). Each vaccine group (e.g., VLP and/or VLP cocktail) or placebos are given at 0, 6, and 20-day intervals, to 10-40 neonatal (8 days) subjects.

VLP Vaccine Cocktail Trial

For a heterogeneous VLP cocktail (e.g., Set VII, Set VIII, and Set IX), the animal model selected is one that is susceptible to multiple viruses (e.g., a heterogeneous VLP cocktail of BIV, BCV, and bovine influenza A virus tested in cattle; a heterogeneous VLP cocktail of SIV, simian virus 40, simian T-cell lymnphotropic virus type 1, and herpes B virus tested in macaque monkeys; etc.). For illustrative purpose only, the monkeys are inoculated IM/IO with 0.3 μg of each VLP of Set IX (as calculated by the surface protein or core/capsid content of each VLP). The first inoculation is followed by two-boost immunizations 10 with 1.0 μg each VLP on day 2, then 10 μg each VLP on day 6. After three inoculations, the subjects are immunized twice with 0.3 μg each VLP at 2-week intervals. Immune responses (VLP-reactive T_{H}, CTL, Ab, and cytokine secretion) are measured. At least one wild type virus (in this case, West Nile virus, LCM virus, Hanta virus, Dengue virus, Ebola virus, and/or Variola virus) is administered to each control and experimental subject at 4 weeks or 3 months following completion of the vaccine regimen. Four weeks after the last vaccination, half of the monkeys are challenged with at least one virus with a dosage of 10³ to 10⁵ TCID₅₀. Three months after the last vaccination, the remaining monkeys are challenged with the same virus at a dosage of 10³ to 10⁵ TCID₅₀. Each vaccine group (e.g., VLP and/or VLP cocktail) or placebos are given at 0, 2, 6, 20, and 34-day intervals, to 10-120 neonatal subjects (1-3 months), 10-120 young (4 month-5 years), and/or 10-120 adolescent and adult subjects (6 years and older).

The timing of vaccine administration is subject to change, as the schedule exemplified above simply corresponds to one embodiment of the present invention. For instance, in other embodiments the vaccine administration protocol is reduced or increased in dosage amount or frequency, or the challenge virus dose or time post vaccination is reduced or increased. In further embodiments, two or more wild type viruses are used for challenge and/or the route of VLP immunization and/or virus challenge is altered (e.g., intratracheal, intravenous, etc.). Additionally, the vaccine schedule varies depending upon the subject species. For example, the life span of a rhesus macaque is approximately 29 years. This is roughly equivalent to a human life span of 80 years. Thus, for human subjects, the vaccine inoculation schedule is lengthened in some embodiments of the present invention.

Molecular adjuvants including, but not limited to the following genes or gene products also find use with the vaccines of the present invention: co-stimulatory molecules (e.g., CD80, CD86), proinflammatory cytokines (e.g., IL-1α, TNF-α, TNF-β), T helper 1 cytokines (e.g., IL-2, IL-12, IL-15 and IL-18), T helper 2 cytokines (e.g. IL-4, IL-5 and IL-10), Flt3 ligand, hematopoietic growth factors (e.g., GM-CSF, SCF), and chemokines (e.g., MIP-1a, MIP-1b, and RANTES). Alternatively, steroids such as methylprednisolone are administrated before or after VLP inoculation. Saline is contemplated to be an appropriate control in these instances. Similarly, a preventive vaccine is suitable for administration to other susceptible species (e.g., VLPs for SARS, hepatitis, encephalitis, and gastroenteritis are suitable for administration to pigs, cattle, and humans; an immunodeficiency virus VLP is suitable for administration to cats, primates, horses, sheep and goats, etc.).

### EXAMPLE 10

### Therapeutic VLP Vaccination Schedule

In some preferred embodiments, vaccines are administered oronasally (IO). However, other routes of vaccine administration (e.g., IM, IV, etc.) find use with the methods and compositions of the present invention. For illustrative purposes, five examples of the present invention are described below.

AIDS VLP Vaccine Trial

The AIDS vaccination schedule summarized here is for therapeutic use in HIV/SIV-seropositive subjects (infected). For a therapeutic protocol, the strategy is to immunize the subjects gradually with an HIV/SIV VLP (therapy dose). In some embodiments, the goal of the vaccine regimen is to bring the infected subject's immune system back to a so-called “autovaccination” state in which the subject's immune system controls an undetectable or barely detectable viral load. In some instances, if an infected subject is unable to reach this state through VLP administration alone, combination therapy with HAART is used. HAART is discontinued after the “autovaccination” state is first reached.

At the start of the experiments, the monkeys are inoculated IO with 30 ng SIV VLP (as calculated by p27 content) on day one. The subjects are immunized at one-week intervals until they reach an “autovaccination” state. For SWV-infected monkeys, this corresponds to a viral load of 10⁴ viral RNA copies/ml or less. Thereafter the monkeys are immunized with the same dose of vaccines once a month for 6 months. After that, they are immunized once every three-months for life. Each vaccine group (e.g., VLP and/or VLP cocktail) qr placebos are given at set intervals, to 10-20 neonatal subjects (e.g., 1-3 months), 10-20 young (4 month-5 years), and/or 10-20 adult and adolescent subjects.

Similarly, trials for infected monkeys with a viral load of 10⁶ viral RNA copies/ml or more are performed using infected rhesus macaques. The monkeys are inoculated 10 with 30 ng SIV VLP (as calculated by p27 content) at day one, along with a combination treatment of HAART. The subjects are immunized at one-week intervals with the SIV VLP vaccine in addition to HAART, until they can control their infections (e.g., undetectable or barely detectable viral load). The HAART regimen is then withdrawn and only the vaccine is used as therapy. HAART is restarted in the case of an uncontrollable rebound of the virus load and/or disease progression. The infected monkeys are immunized with the same dose of vaccine until they can achieve an undetectable viral load. They are immunized with the same dose of vaccine once a month for 6 months. After that, immunizations are given at intervals of once every three-month for life. Each vaccine group (e.g., VLP and/or VLP cocktail) or placebos are given at set intervals, to 10-20 neonatal subjects (1-3 months), 10-20 young (4 month-5 years), and/or 10-20 adolescent and adult subjects (6 years and older).

SARS VLP Vaccine Trial

The exemplary SARS VLP vaccination schedule provided here is for therapeutic use in SCV-seropositive subjects (infected). An SCV VLP or VLP cocktail (e.g., Set II and Set V) is utilized. At the start of the experiment, cats are inoculated IO with 30 ng SCV VLP vaccine (as calculated by N protein content) at day one. The subjects are immunized at one-week intervals until they can control their viral load (e.g., achieve an undetectable viral load). Then they are boosted with the same dose of SCV VLP vaccines once a month for 6 months. After that, they are immunized once every three-months for life. Each vaccine group (e.g., VLP and/or VLP cocktail) or placebos are given at set intervals, to 10-20 cats (20 weeks and older) subjects.

HCV VLP Vaccine Trial

The hepatitis C virus VLP vaccination schedule provided here is for therapeutic use in hepatitis C virus type-seropositive subjects (infected). An HCV VLP or VLP cocktail (e.g., HCV-1, HCV-H, and HCV-J1 as a homologous cocktail or HCV-1a, HCV-2b, and HCV-3a as a heterogeneous cocktail) is utilized. At the start of the trial, SPF chimpanzees (Pan troglodytes) are inoculated IM with 50 ng HCV VLP (as calculated by core protein content) at day one. The subjects are immunized at one-week intervals until they can achieve an undetectable viral load. Then they are immunized with the same dose of HCV VLP vaccines once a month for 6 months. After that, they are immunized once every three-months for life. Each vaccine group (e.g., VLP and/or VLP cocktail) or placebos are given at set intervals, to 3-12 subjects.

VLP Vaccine Cocktail Trial

For a heterogeneous VLP cocktail (e.g., Set VII, Set VIII, and Set IX), the animal model selected is one that is susceptible to infection with multiple viruses. For illustrative purposes, monkeys infected with SIV, simian virus 40, simian T-cell lymphotropic virus type 1, herpes B virus, and/or measles virus, are selected as subjects. A VLP vaccine cocktail comprising a heterogeneous mixture of SIV, simian virus 40, simian T-cell lymphotropic virus type 1, herpes B virus, and measles virus VLP vaccines is administered in a single IM or IO inoculation containing 30 ng of each VLP (as calculated by the structural protein or surface antigen content) on day one. The subjects are boosted at one-week intervals, and the presence of each virus is monitored. When the subjects have achieved an undetectable viral load for all virus infections, the immunization schedule is reduced to once a month for 6 months. After that, the subjects are immunized once every three-months for life. If necessary, a recombinant drug therapy comprising antiviral medications is combined with this regimen. Each vaccine group (e.g., VLP and/or VLP cocktail) or placebos are given to 10-120 neonatal subjects (1-3 months), 10-120 young (4 month-5 years), and/or 10-120 adolescent or adult subjects (6 years and older).

The VLP vaccine cocktail is subject to change depending upon the infection status of the study groups. For instance, the administration protocol of vaccines is altered if a subject is infected by a virus that can be cleared from the subject as determined at a selected period of time post vaccination or certain time (e.g., influenza virus infections). In contract, a subject infected by HIV, HCV, and SCV is expected to remain chronically infected with the three viruses for life. Thus, a VLP vaccine regimen comprising an HIV, HCV, and SCV VLP vaccine cocktail is administrated to a subject indefinitely. Additionally, the vaccine schedule varies depending upon the species of the subject and/or the extent of disease progression. For example, for a subject with a double infection (e.g., RSV and HIV), the vaccination protocol begins with a cocktail of both RSV and HIV VLPs. If and when the RSV infection is cleared, only the HIV VLP vaccine is administered.

### EXAMPLE 11

### Viral Challenge

Controls are utilized during testing of the VLP methods and compositions of the present invention. In an exemplary embodiment, a total of four different protocols are utilized to test the efficacy of the VLP vaccines described in Example 9. The first protocol comprises an experimental group receiving the AIDS VLPs of Set I and Set IV, and a control group receiving a placebo (e.g., saline, vector without viral gene(s) of interest, adjuvant, etc). The second protocol comprises an experimental group receiving the SARS VLPs of Sets II and Set V, and a control group receiving a placebo. The third protocol comprises an experimental group receiving the Influenza VLPs of Set III and Set VI, and a control group receiving a placebo. The fourth protocol comprises an experimental group receiving a heterogeneous VLP cocktail of Set IX (e.g., for the purpose of assessing protection from a bioterrorist weapon) and a control group receiving a placebo.

Likewise, multiple different protocols are utilized to test the efficacy of the VLP vaccines described in Example 10. The first protocol comprises and experimental group receiving the AIDS VLPs of Set I and a control group receiving a placebo with HAART only as needed. The second protocol comprises an experimental group receiving the AIDS VLPs of Set I and a control group receiving a placebo in addition to HAART. The third protocol comprises an experimental group receiving the SCV VLPs and a control group receiving a placebo, with or without SARS drug treatment. The third protocol comprises an experimental group receiving the HCV VLPs and a control group receiving a placebo, with or without HCV drug treatment. The fifth protocol comprises an experimental group receiving a VLP cocktail (e.g., SIV, simian virus 40, simian T-cell lymphotropic virus type 1, herpes B virus, and measles virus VLPs) and a control group receiving a placebo, with or without antiviral drug treatment. Additional protocols are also contemplated to be within the scope of the present invention. Each group comprises about 3 to 40 subjects.

A live virus challenge is used to test the efficacy of the VLP immunogens of the present invention in a suitable model. For illustrative purpose only, a SIV live virus challenge is used to test the efficacy of the AIDS VLP Vaccines of the present invention in the rhesus monkey model. In particular, wild type SIV_{mac239} or SIV_{mac251} is administered to each control and experimental subject at 4 weeks and/or 3 months following completion of the AIDS VLP vaccine regimen. In another embodiment, an avian influenza A live virus challenge is used to test the efficacy of the Influenza A VLP Vaccines of the present invention in the chicken model. In particular, wild type A/PR/8/34 (HINI) or A/CK/HK/86.3/02 (H5N1) is administered to each control and experimental subject 4 weeks following completion of the avian influenza A vaccine regimen. In a further embodiment a SCV live virus challenge is used to test the efficacy of the SARS VLP Vaccines of the present invention in the ferret model. Specifically, wild type SCV HK-39 is administered to each control and experimental subject 4 weeks and/or 3 months following completion of the SARS vaccine regimen. Other live virus challenge protocols known in the art are also suitable including but not limited to: HIV as described by Miller et al., J Virol, 71:1911-21, 1997; and Lena et al., Vaccine, 20 Suppl 4:A69-79, 2002; HCV as described by Puig et al., Vaccine, 22:991-1000, 2004; and Rollier et al., J Virol, 78:187-96, 2004; SCV and coronavirus as described by Gao et al., Lancet, 362:1895-6, 2003; and Glansbeek et al., J Gen Virol, 83:1-10, 2002; influenza A virus as described by Liu et al., Virology, 314:580-90, 2003; and Van Reeth et al., Vaccine, 21:1375-81.2003. Each control and experimental protocol is divided into 3 sections (e.g., A, B, and C) consisting of 2 to 10 subjects each. In a preferred embodiment, each subject is challenged through the IO route. However, with such a diversity of viruses and animal models, the route of vaccine administration is subject to change. For illustrative purpose only, in the AIDS vaccine model, adult male subjects are challenged with a dosage of 10³ TCID₅₀ via the intravenous route, adult female subjects are challenged with a dosage of 10⁵ TCID₅₀ via the intravaginal route, and youth subjects are challenged with a dosage of 10⁵ TCID₅₀ via the oral/nasal route, at week 4 (section A), month 3 (section B), or month 6 (section C).

It is contemplated that subjects of some experimental protocols, are protected from viral challenge. Such regimens are farther contemplated to be suitable for protecting human subjects from virus infection and/or disease onset. For illustrative purpose only, it is contemplated that subjects of the experimental group of the first protocol (receiving VLPs of Example 9) are protected from SIV viral infection and similarly that this regimen is suitable for protecting humans from HIV infection. Likewise, it is contemplated that the subjects of the second protocol (receiving VLPs of Example 9) are protected from FIPV infection and similarly that this regimen is suitable for protecting humans from SCV infection. Further, it is contemplated that the subjects of the experimental group of the third protocol (receiving VLPs of Example 9) are protected from avian influenza A viral infection, and similarly that this regimen is suitable for protecting humans from human influenza A virus infection. In contrast, control protocols are contemplated to offer no protection from challenge.

Additionally it is contemplated that subjects of the experimental group of the first protocol (receiving SIV VLPs of Example 10) attain a reduction in immunodeficiency virus load infection and a delay in AIDS development. Furthermore, it is contemplated that subjects of the experimental group of the second protocol (receiving FIPV VLPs of Example 10) are cured from FIPV viral infection and similarly that this regimen is suitable for curing SCV-infected humans of SARS. Lastly, it is contemplated that subjects of the experimental group of the third protocol (receiving HCV VLPs of Example 10) are cured from HCV infection and similarly that this regimen is suitable for delaying hepatitis and hepatocellular carcinoma progression in HCV-infected humans.

### EXAMPLE 12

### Immune Assays

The VLP vaccine methods and compositions described herein are contemplated to induce both virus (e.g., HIV, HCV, SCV, HTLV, influenza viruses, etc.) specific humoral and cellular immune responses. One day prior to each experimental or placebo vaccination, serum samples are collected for analysis. Humoral assays such as the measurement of neutralizing antibodies are performed as described: for HIV assays see Shacklett et al., J Virol, 76:11365-78, 2002; and Letvin et al., J Virol, 78:7490-7, 2004; for HCV assays see Logvinoff et al., Proc Natl Acad Sci USA, 101:10149-54, 2004; for SCV and coronavirus assays see Woo et al., J Clin Microbiol, 42:2306-9, 2004; and de Haan et al., Virology, 296:177-89, 2002; for influenza A virus assays see de Jong et al., Dev Biol (Basel), 115:63-73, 2003; and Ali et al., Clin Infect Dis, 38:760-2, 2004. T cell responses including the detection of Thl and Th2 cytokines (e.g., IL-2, IL-4, IL-10, INF-γ, etc.) are measured by cytokine ELISA or ELISPOT. Additionally, CTL assays, CD4/CD8 ratios and lymphocyte proliferation assays are performed as described: for HIV see Mooij et al., J Virol, 78:3333-42, 2004; and Deml et al., Methods Mol Med, 94:133-57, 2004; for HCV see Nascimbeni et al., J Virol, 77:4781-93, 2003; and Jiao et al., J Gen Virol, 85:1545-53, 2004; for SCV and coronavirus see Zhu et al., Immunol Lett, 92:237-43, 2004; and Glansbeek et al., J Gen Virol, 83:1-10, 2002; for influenza A virus see Chen et al., Virus Res, 103:147-53, 2004; and Babiuk et al., J Med Virol, 72:138-42, 2004.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in the relevant fields, are intended to be within the scope of the following claims.

1. A method for producing a virus-like particle, comprising the steps of:
a) providing:
i) an encapsidation system comprising at least one packaging vector and two or more helper vectors, wherein said packaging vector comprises a nucleic acid having a packaging signal(s) but lacking an integration signal(s), and wherein said helper vectors each comprise one or more viral genes encoding one or more viral proteins, and
ii) a cell line;
b) introducing said encapsidation system into said cell line under conditions suitable for causing expression of said nucleic acid as a packaging signal and said one or more viral genes as one or more viral proteins, to produce a virus-like particle comprising one or more viral proteins assembled around said packaging signal; and
c) purifying said virus-like particle.

2. The method of claim 1, wherein said packaging vector further comprises a promoter/enhancer, and a terminator or polyadenylation site.

3. The method of claim 2, wherein said encapsidation vector further comprises a viral capsid gene in operable combination with said promoter/enhancer and said terminator or polyadenylation site.

4. The method of claim 1, wherein said at least two helper vectors comprises a first helper vector comprising a viral envelope or other structural protein gene, and second helper vector comprising a viral polymerase or other nonstructural gene.

5. The method of claim 4, wherein said at least two helper vectors further comprise one or more of a viral capsid gene, a viral regulatory gene, and a viral accessory gene.

6. The method of claim 1, wherein said virus-like particle is replication-deficient.

7. The method of claim 6, wherein said virus-like particle is attachment and penetration competent.

8. The method of claim 4, wherein penetration comprises fusion or endocytosis.

9. The method of claim 4, wherein said virus-like particle is an immunodeficiency type virus-like particle.

10. The method of claim 9, wherein said immunodeficiency type virus-like particle is an HIV-1 or HIV-2 virus-like particle.

11. The method of claim 1, wherein said encapsidation system comprises at least three helper vectors comprising a first helper vector comprising a viral spike gene, a second helper vector comprising a viral replicase gene, and a third helper vector comprising one or more viral subgenomic genes.

12. The method of claim 1l, wherein said virus-like particle is a coronavirus type virus-like particle.

13. The method of claim 12, wherein said coronavirus type virus-like particle is an SCV virus-like particle.

14. The method of claim 4, wherein said virus-like particle is a hepatitis type virus like particle.

15. The method of claim 1, wherein said encapsidation system comprises at least eight helper vectors comprising a first helper vector having a viral polymerase A gene, a second helper vector having a viral polymerase B1 gene, a third helper vector comprising a viral polymerase B2 gene, a fourth helper vector comprising a viral nucleoprotein gene, a fifth helper vector comprising a viral membrane protein gene, a sixth helper vector comprising one or more viral nonstructural protein genes, a seventh helper vector comprising a viral hemagglutinin gene lacking a packaging signal and an eighth helper vector comprising a viral neuraminadase gene lacking a packaging signal.

16. The method of claim 1, wherein said encapsidation system comprises a first packaging vector comprising a nucleic acid encoding a hemagglutinin packaging signal and a second packaging vector comprising a nucleic acid encoding a neuraminadase packaging signal.

17. The method of claim 16, wherein said virus-like particle is an influenza type virus-like particle.

18. A kit for producing of a virus-like particle, comprising:
a) an encapsidation system comprising at least one packaging vector and two or more helper vectors, wherein said packaging vector comprises a nucleic acid having a packaging signal(s) but lacking an integration signal(s), and said at helper vectors each comprise one or more viral genes encoding one or more viral proteins, and
b) instructions for cloning one or more viral genes lacking packaging signals into said helper vectors, and for contacting a suitable cell line with said encapsidation system under conditions suitable for causing expression of said nucleic acid as a packaging signal and said one or more viral genes as one or more viral proteins, to produce a virus-like particle comprising one or more viral proteins assembled around said packaging signal.

19. The kit of claim 18, further comprising said suitable cell line.

20. The kit of claim 18, wherein said encapsidation vector further comprises a promoter/enhancer, and a terminator or polyadenylation site.

21. A method, comprising:
a) providing:
i) a subject; and
ii) a composition comprising the virus-like particle of claim 1; and
b) administering said composition to said subject under conditions such that an immune response reactive with said virus-like particle is generated.

22. The method of claim 21, wherein said immune response comprises one or more of a lymphocyte proliferative response, cytokine response, cytotoxic T lymphocyte response and antibody response.

23. The method of claim 22, wherein said cytokine response comprises secretion of one or more of an interleukin, interferon, tumor necrosis factor, chemokine, and growth factor.

24. The method of claim 22, wherein said antibody response comprises production of IgG antibodies and/or IgA antibodies.

25. The method of claim 21, wherein said subject is a mammal selected from the group consisting of a human, a nonhuman primate, a horse, a cow, a sheep, a rodent, a goat and a cat.

26-30. (canceled)
